(19) United States
(12) Patent Application Publication
(10) Pub. No.: US 2011/0251194 A1
(43) Pub. Date: Oct. 13, 2011

(54) ANTINEOPLASTIC DERIVATIVES, PREPARATION THEREOF AND THERAPEUTIC USE THEREOF

(75) Inventors: Claude BERNHART, Saint Gely du Fesc (FR); Gary MCCORT, Paris (FR); Samir JEGHAM, Montferrier-sur-lez (FR); Jean Marc HERBERT, Tournefeuille (FR); Pierre CASELLAS, Castelnau-le-lez (FR); Monsif BOUABOULA, Juvignac (FR); Olivier DUCLOS, Paris (FR)

(73) Assignee: SANOFI-AVENTIS, Paris (FR)

(21) Appl. No.: 12/986,642

(22) Filed: Jan. 7, 2011

Related U.S. Application Data
(63) Continuation of application No. PCT/FR2009/051322, filed on Jul. 6, 2009.

(30) Foreign Application Priority Data
Jul. 8, 2008 (FR) 0803863

(51) Int. Cl. A61K 31/496; C07D 401/02; A61K 31/5377; A61K 31/4709; A61P 35/00; C07D 471/04; A61K 31/444

(52) U.S. Cl. 514/234.5; 546/123; 544/357; 546/156; 514/300; 514/252.11; 514/312; 544/127

(57) ABSTRACT

The disclosure concerns heterobicyclic compounds of general formula (I) and acid addition salts, hydrates and solvates thereof, as well as enantiomers, diastereoisomers and mixtures thereof. Methods for preparing the compounds, pharmaceutical compositions, and methods of treatment also are disclosed.

### CROSS-REFERENCE TO RELATED APPLICATIONS

This application is a continuation of International application No. PCT/FR2009/051322, filed Jul. 6, 2009, which claims the benefit of priority of French Patent Application No. 0803863, filed Jul. 8, 2008, both of which are incorporated herein by reference.

### BACKGROUND

The present invention relates to anticancer derivatives, to the compositions containing them and to the therapeutic use thereof. The invention also relates to the process for preparing these compounds and also to some of the intermediate products used.

Application WO 2005/073229 describes compounds of formula (A) in which R₅ represents a hydrogen atom, F, Cl, Br, OH, NO₂, CN, or a (C₁-C₆)alkyl or O—(C₁-C₆)alkoxy group optionally substituted with F, Cl or Br:

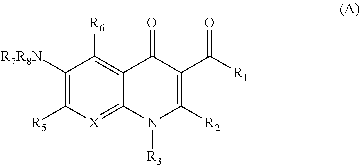

Application EP 0978516 describes compounds of formula (B) in which R₄ represents a hydrogen atom, an optionally substituted alkyl group or a halogen atom:

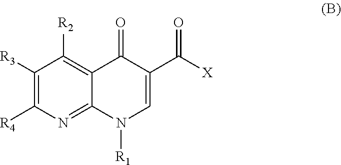

Japanese application JP 4077488 describes compounds of formula (C) in which X₁ represents a halogen atom and R₃ represents a halogen atom or an alkoxy, alkylthio, alkylsulfinyl, alkylsulfonyl, arylthio, arylsulfinyl or arylsulfonyl group:

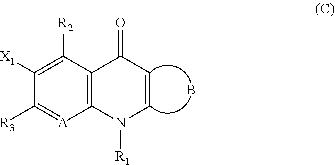

U.S. Pat. No. 5,622,967 describes compounds of formula (D):

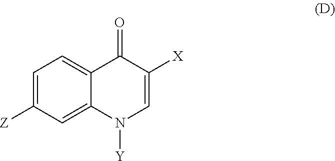

in which Z represents an optionally substituted aryl or heteroaryl group, a phenylalkyl group, a heterocycloalkyl group, etc.

Application WO 2006/016067 describes pyridopyrimidine derivatives of formula (E):

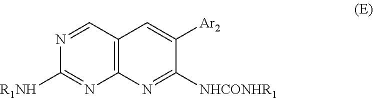

the nucleus of which is different from the nucleus of the compounds of the invention.

None of these documents describes the compounds of the invention.

### DETAILED DESCRIPTION

### Definitions Used

In the context of the present invention, and unless otherwise mentioned in the text:
the term “a halogen atom” is intended to mean: a fluorine, chlorine, bromine or iodine atom (advantageously fluorine);
the term “an alkyl group” is intended to mean: a linear or branched, saturated aliphatic hydrocarbon-based group containing from 1 to 6 carbon atoms (advantageously from 1 to 4 carbon atoms), obtained by removing a hydrogen atom from an alkane. By way of examples, mention may be made of methyl, ethyl, propyl, isopropyl, butyl, isobutyl, tert-butyl, pentyl, 2,2-dimethylpropyl or hexyl groups;
the term “an alkoxy group” is intended to mean: an —O-alkyl group, where the alkyl group is as defined above;
the term “a heteroatom” is intended to mean: a nitrogen, oxygen or sulfur atom;
the term “a cycloalkyl group” is intended to mean: a cyclic alkyl group containing between 3 and 8 carbon atoms involved in the cyclic structure. By way of examples, mention may be made of cyclopropyl, cyclobutyl, cyclopentyl or cyclohexyl groups;
the term “an aryl group” is intended to mean: a monocyclic or bicyclic, aromatic group containing from 6 to 10 ring members, for example a phenyl or naphthyl group;
the term “a heteroaryl group” is intended to mean: a monocyclic, bicyclic or tricyclic aromatic group comprising between 5 and 14 ring members and comprising one or more heteroatom(s);
the term “a heterocycloalkyl group” is intended to mean: a cycloalkyl group as defined above, additionally comprising 1 to 4 heteroatoms involved in the cyclic structure. By way of example, mention may be made of pyrrolidinyl, piperidinyl, tetrahydrofuryl, morpholinyl and piperazinyl groups;
the term “protective group” is intended to mean: a group intended to protect a chemical function against unwanted chemical reactions, which is introduced over the course of a protection stage and which is released over the course of a subsequent stage. Examples of protective groups will be found in T. W. Greene et al., “Protective Groups in Organic Synthesis”, 3^{rd} edition, 1999, Wiley-Interscience or in J. F. W. McOmie, “Protective Groups in Organic Chemistry”, Plenum Press, 1973;
the term “optionally substituted” is intended to mean the fact that each of the preceding groups may be substituted with one or more groups, which may be identical to or different from one another, chosen from: hydroxyl, (C₁-C₆)alkoxy, preferably (C₁-C₄)alkoxy, (C₁-C₆)alkyl, preferably (C₁-C₄)alkyl, or a halogen atom.

According to a 1^{st} aspect, a subject of the present invention is a compound of formula (I):

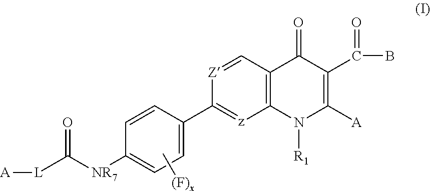

in which:
R₁ represents:
   a (C₃-C₇)cycloalkyl group or
   a (C₁-C₆)alkyl group optionally substituted with an —NRₐR_{b}
group in which:
   (i) Rₐ and R_{b} represent, independently of one another, a hydrogen atom or a (C₁-C₄)alkyl group, or
   (ii) Rₐ and R_{b} form, together with the nitrogen atom to which they are attached, a heterocycloalkyl group which optionally comprises another heteroatom in the ring and which is optionally substituted;
   or (iii) Rₐ represents a hydrogen atom and R_{b} represents a —C(═O)O(C₁-C₄)alkyl group;
A represents an —NR₂R₃ group;
B represents an —NR₄R₅ or —OR₆ group;
R₂ and R₃ are such that:
   R₂ and R₃ represent, independently of one another, a hydrogen atom or a (C₁-C₆)alkyl group;
   or R₂ represents a hydrogen atom and R₃ represents a (C₁-C₆)alkyl group substituted with:
      an optionally substituted heterocycloalkyl group;
      an —NR_{c}R_{d} group in which:
      (i) R_{c} and R_{d} represent, independently of one another, a hydrogen atom or a (C₁-C₄)alkyl group;
      or (ii) R_{c} and R_{d} form, together with the nitrogen atom to which they are attached, a heterocycloalkyl group which optionally comprises another heteroatom in the ring and which is optionally substituted;
R₄ and R₅ are such that:
   R₄ and R₅ represent, independently of one another, a hydrogen atom or a (C₁-C₄)alkyl group, or form, together with the nitrogen atom to which they are attached, an optionally substituted heterocycloalkyl group;
   or R₄ represents a hydrogen atom and R₅ represents a (C₁-C₆)alkyl group substituted with:
      an optionally substituted heterocycloalkyl group;
      an —NRₑR_{f} group in which:
      (i) Rₑ and R_{f} represent a hydrogen atom or a (C₁-C₄)alkyl group;
      or (ii) Rₑ and R_{f} form, together with the nitrogen atom to which they are attached, a heterocycloalkyl group which optionally comprises another heteroatom in the ring and which is optionally substituted;
      or (iii) Rₑ represents a hydrogen atom and R_{f} represents a —C(═O)O(C₁-C₄)alkyl group;
   or R₄ represents a hydrogen atom and R₅ represents one of the following groups: —C(CH₂OH)₃; —[(CH₂)₂O]ₘCH₂NH₂ or —[(CH₂)₃NH]ₘ—H in which m is an integer ranging from 3 to 10;
with the condition that, if R₂ represents a hydrogen atom and R₃ represents a (C₁-C₆)alkyl group substituted with a heterocycloalkyl group or an —NR_{c}R_{d} group, then R₄ and R₅ are respectively identical to R₂ and R₃;
R₆ represents a hydrogen atom or a (C₁-C₄)alkyl group;
Z represents N or CH;
Z′ represents N or CH if Z represents N, and CH if Z represents CH;
x is an integer ranging from 0 to 4;
R₇ represents a hydrogen atom or a (C₁-C₄)alkyl group;
L represents a —CH═CH—, —CH₂CH₂—, —CH₂CH[NHC(═O)O(C₁-C₄)alkyl)] or —(CH₂)ₙ—Y— group in which n is an integer ranging from 1 to 4 and the group Y (connected to C═O) represents an oxygen atom or an —NR₈— group in which R₈ represents a hydrogen atom or a (C₁-C₄)alkyl group;
Ar represents a group chosen from:

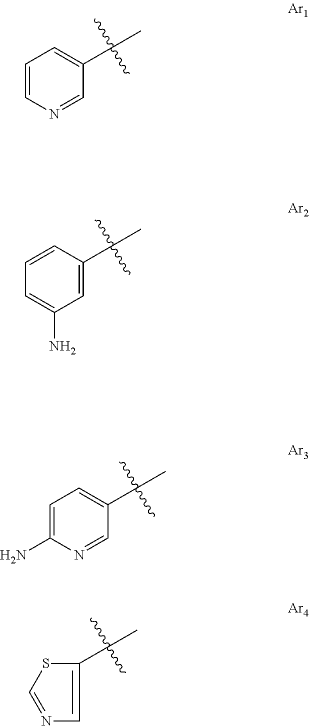

R₁ represents a (C₃-C₇)cycloalkyl group or a (C₁-C₆)alkyl group optionally substituted with an —NRₐR_{b} group. Rₐ and R_{b} can represent, independently of one another, a hydrogen atom or a (C₁-C₄)alkyl group.

Rₐ and R_{b} may also form, together with the nitrogen atom to which they are attached, a heterocycloalkyl group which optionally comprises another heteroatom in the ring and which is optionally substituted. This heterocycloalkyl group may, for example, be the 4-morpholinyl

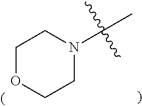

or pyrrolidinyl

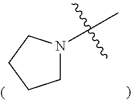

group.

Rₐ may also represent a hydrogen atom and R_{b} a —COO(C₁-C₄)alkyl group, for example the —COOtBu group.

R₁ may be one of those of Table I.

A may represent an —NR₂R₃ group.

R₂ and R₃ may represent, independently of one another, a hydrogen atom or a (C₁-C₆)alkyl group. R₂ and R₃ may both represent a hydrogen atom, or else R₂ may represent a hydrogen atom and R₃ a (C₁-C₆)alkyl group. R₂ may also represent a hydrogen atom and R₃ a (C₁-C₆)alkyl group substituted with an optionally substituted heterocycloalkyl group, such as, for example, the 4-piperidinyl

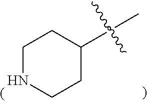

### or 4-N-alkyl-piperidinyl

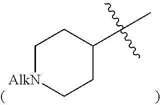

group, for example 4-N-methylpiperidinyl, or 2-tetrahydrofuryl

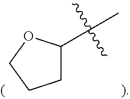

R₂ may also represent a hydrogen atom and R₃ a (C₁-C₆)alkyl group substituted with an —NR_{c}R_{d} group. R_{c} and R_{d} may represent, independently of one another, a hydrogen atom or a (C₁-C₄)alkyl group. R_{c} and R_{d} may also form, together with the nitrogen atom to which they are attached, a heterocycloalkyl group which optionally comprises another heteroatom in the ring and which is optionally substituted: for example, the 4-morpholinyl group

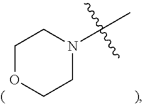

pyrrolidinyl group

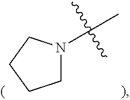

piperazinyl group

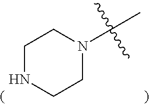

or N-alkylpiperazinyl group

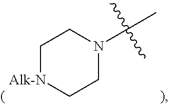

for example N-methylpiperazinyl, or piperidinyl group

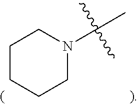

As examples of substituted heterocycloalkyl groups, mention may be made of the following groups: 2-methylpyrrolidinyl

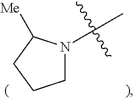

4-hydroxypiperidinyl

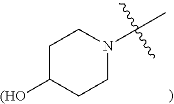

or 4,4′-difluoropiperidinyl

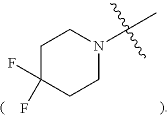

R₂ and R₃ and also the R₂/R₃ combinations may be chosen from Table I.

B may represent an —NR₄R₅ group.

R₄ and R₅ may represent, independently of one another, a hydrogen atom or a (C₁-C₆)alkyl group. R₄ and R₅ may both represent a hydrogen atom or else a (C₁-C₆)alkyl group, or else R₄ may represent a hydrogen atom and R₅ a (C₁-C₆)alkyl group.

R₄ and R₅ may also form, together with the nitrogen atom to which they are attached, an optionally substituted heterocycloalkyl group, such as the piperazinyl

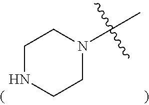

### or N-alkylpiperazinyl

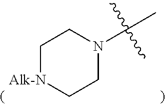

group, for example N-methylpiperazinyl.

R₄ may represent a hydrogen atom and R₅ a (C₁-C₆)alkyl group substituted with an optionally substituted heterocycloalkyl group, such as, for example, the 4-piperidinyl or 4-N-alkylpiperidinyl group, for example 4-N-methylpiperidinyl or 2-tetrahydrofuryl.

R₄ may represent a hydrogen atom and R₅ a (C₁-C₆)alkyl group substituted with an —NRₑR_{f} group. Rₑ and R_{f} may represent a hydrogen atom or a (C₁-C₄)alkyl group.

Rₑ and R_{f} may also form, together with the nitrogen atom to which they are attached, a heterocycloalkyl group which optionally comprises another heteroatom in the ring and which is optionally substituted; for example, the 4-morpholinyl group, pyrrolidinyl group, piperazinyl group or N-alkylpiperazinyl group, for example N-methylpiperazinyl group, or piperidinyl group. As examples of substituted heterocycloalkyl groups, mention may be made of the following groups: 2-methylpyrrolidinyl, 4-hydroxypiperidinyl or 4,4′-difluoropiperidinyl.

Rₑ may also represent a hydrogen atom and R_{f} a —COO(C₁-C₄)alkyl group, such as the —COOtBu group.

Finally, R₄ may represent a hydrogen atom and R₅ one of the following groups: —C(CH₂OH)₃; —[(CH₂)₂O]ₘCH₂NH₂ or —[(CH₂)₃NH]ₘ—H in which m is an integer ranging from 3 to 10.

R₄ and R₅ and also the R₄/R₆ combinations may be chosen from Table I.

B may represent an —OR₆ group in which R₆ represents a hydrogen atom or a (C₁-C₄)alkyl group. R₆ may be chosen from Table I.

Z represents N or CH and Z′ represents N or CH if Z represents N, and CH if Z represents CH. The ring comprising Z and Z′ is therefore one of the following 3 rings:

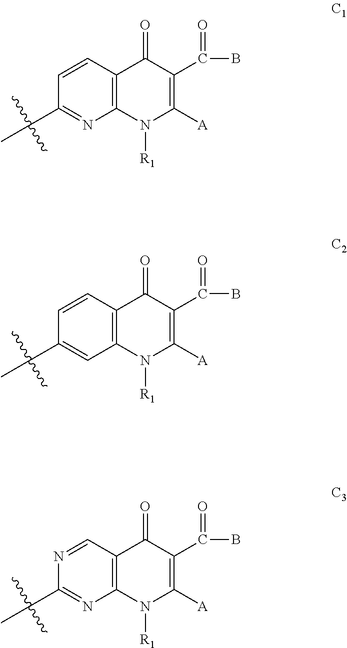

x represents the number of fluorine atom(s) as substituent of the ring; this integer ranges from 0 (no fluorine atom) to 4.

R₇ represents a hydrogen atom or a (C₁-C₄)alkyl group. Preferably, it is a hydrogen atom. R₇ may be chosen from Table I.

L represents a —CH═CH—, —CH₂CH₂—, —CH₂CH[NHC(═O)O(C₁-C₄)alkyl]- or —(CH₂)ₙ—Y— group in which n is an integer ranging from 1 to 4 and the group Y (connected to C═O) represents an oxygen atom or an —NR₈— group in which R₈ represents a hydrogen atom or a (C₁-C₄)alkyl group. Preferably, when L represents a —(CH₂)ₙ—NR₈— group, at least one of the two groups R₇ or R₈ is a hydrogen atom. Preferably, the two groups R₇ or R₈ represent a hydrogen atom (i.e. the unit linking Ar and the phenyl ring is —(CH₂)ₙ—NH—C(═O)—NH—).

L may be one of those described in table I. Preferably, L represents the —CH₂—NH—, —CH₂—O— or —CH═CH— group. When L represents the —CH═CH— group, preference is also given to the E isomers rather than the Z isomers.

Ar represents a group chosen from:

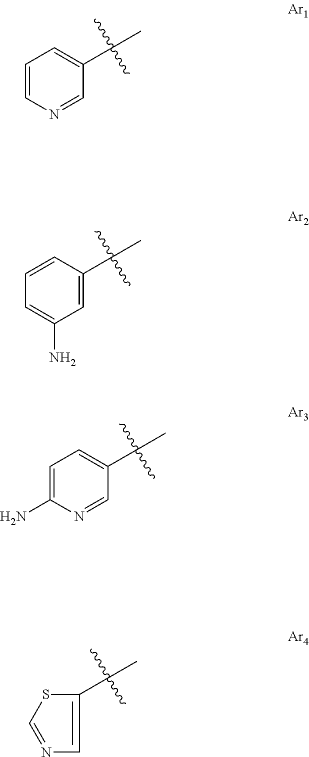

A 1^{st} subgroup is differentiated, for which:
R₁ represents a (C₁-C₆)alkyl group;
A represents an —NHR₃ group in which R₃ represents a hydrogen atom or a (C₁-C₄)alkyl group;
B represents an —NR₄R₅ group in which R₄ represents a hydrogen atom or a (C₁-C₆)alkyl group and R₅ represents:
   a hydrogen atom;
   a (C₁-C₆)alkyl group optionally substituted with:
      an optionally substituted heterocycloalkyl group;
      an —NRₑR_{f} group as defined above;
   or one of the following groups: —C(CH₂OH)₃; —[(CH₂)₂O]ₘCH₂NH₂ or —[(CH₂)₃NH]ₘ—H in which m is an integer ranging from 3 to 10.

In this 1^{st} subgroup, the compounds for which R₃ and/or R₄ represent a hydrogen atom can be differentiated.

A 2^{nd} subgroup can also be differentiated, for which:
R₁ represents a (C₁-C₆)alkyl group substituted with an —NRₐR_{b} group as defined above;
A represents an —NH₂ group;
B represents an —NR₄R₅ group in which R₄ represents a hydrogen atom or a (C₁-C₆)alkyl group.

A 3^{rd} subgroup can also be differentiated, for which:
R₁ represents a (C₁-C₆)alkyl group;
A and B represent an —NHR₃ group in which R₃ represents a (C₁-C₆)alkyl group substituted with a heterocycloalkyl group or with an —NR_{c}R_{d} group as defined above.

The compounds of the 3^{rd} subgroup therefore have general formula (II):

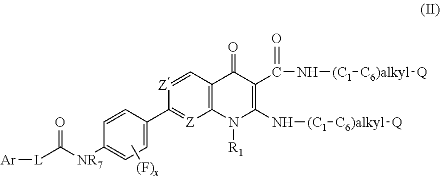

in which Q denotes an optionally substituted heterocycloalkyl group or else the —NR_{c}R_{d} group. The heterocycloalkyl group may be more particularly the morpholinyl group, pyrrolidinyl group, piperazinyl group or N-alkylpiperazinyl group, for example N-methylpiperazinyl group, piperidinyl group, 2-methylpyrrolidinyl group, 4-hydroxypiperidinyl group, 4,4′-difluoropiperidinyl group or 2-tetrahydrofuryl group.

For all these subgroups, Ar, L, R₇, R₈, Z, Z′ and x have the same meanings as above. More particularly, for the compounds of formula (I) and also for the subgroups:
Ar represents the Ar₁ group;
and/or R₇ represents a hydrogen atom;
and/or L represents CH₂NH;
and/or Z and Z′ represent respectively N and CH.

Even more particularly, Ar=Ar₁; R₇=H; L=CH₂NH; Z=N; Z′=CH.

Among the compounds which are subjects of the invention, mention may more particularly be made of the compounds of Table I.

The compounds of the invention may exist in the form of bases or of addition salts with an acid. These salts are advantageously prepared with pharmaceutically acceptable acids, but the salts of other acids that are useful, for example, for purifying or isolating the compounds are also part of the invention. The compounds according to the invention may also exist in the form of hydrates or of solvates, i.e. in the form of associations or combinations with one or more molecules of water or with solvent.

The compounds may optionally comprise one or more asymmetrical carbon atoms and may therefore exist in the form of enantiomers and/or diastereoisomers, or of mixtures of these forms.

According to a 2^{nd} aspect, a subject of the invention is the process for preparing the compounds of the invention and also some of the reaction intermediates.

### Preparation of the Compounds of Formula (I) or (II)

The compounds of formula (I) are obtained according to either of Schemes 1 and 2, it being understood that, in the particular case of the compounds of formula (II), only Scheme 2 using P₃ applies.

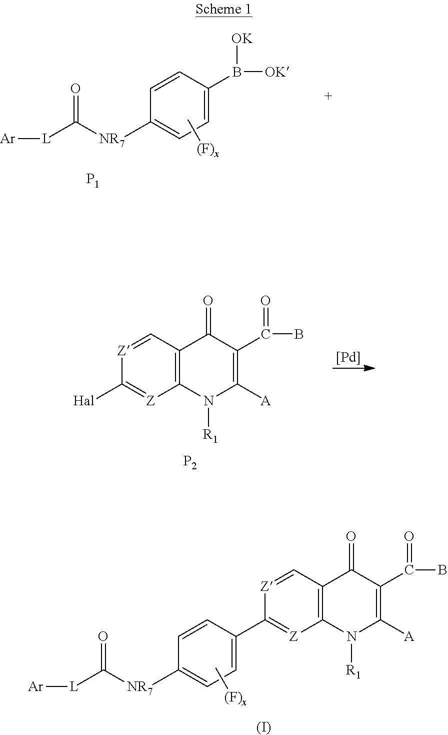

The compound of formula (I) is obtained according to Scheme 1 by Suzuki coupling of P₁ and P₂. Hal represents a halogen atom (chlorine, bromine, iodine). When Z and Z′ both represent CH, the coupling is promoted if Hal is a bromine or iodine atom. When Z and Z′ both represent N or respectively N and CH, Hal may be a chlorine atom. The coupling is carried out in the presence of a palladium (in the oxidation state (0) or (II)) complex such as, for example, Pd(PPh₃)₄, PdCl₂(PPh₃)₂, Pd(OAc)₂ or PdCl₂(dppf). The complexes most commonly used are palladium(0) complexes. The coupling is also promoted in the presence of a base, which may be, for example, K₂CO₃, NaHCO₃, Et₃N, K₃PO₄, Ba(OH)₂, NaOH, KF, CsF, Cs₂CO₃, etc. The coupling may be carried out in a mixture of an ethereal solvent and an alcohol, for example a dimethoxyethane (DME)/ethanol mixture. The temperature at which the reaction is carried out may be between 50 and 120° C.

Further details on the Suzuki coupling, on the operating conditions and also on the palladium complexes that can be used will be found in: N. Miyaura and A. Suzuki, Chem. Rev. 1995, 95, 2457-2483; A. Suzuki in “Metal-catalyzed cross-coupling reactions”; Diederich, F.; Stang, P. J. Eds. Wiley-VCH: Weinhein, Germany, 1998, chap. 2, 49-97; Littke, A. and Fu, G., Angew. Chem. Int., Ed. 1999, 38, 3387-3388 and Chemler, S. R. Angew. Chem. Int. Ed. 2001, 40, 4544-4568.

K and K′ represent a hydrogen atom, or an alkyl or aryl group, optionally linked to one another so as to form, together with the boron atom and the two oxygen atoms a 5- to 7-membered ring optionally substituted with one or more alkyl group(s) or optionally joined to a phenyl group. For example, one of the following groups may be used:

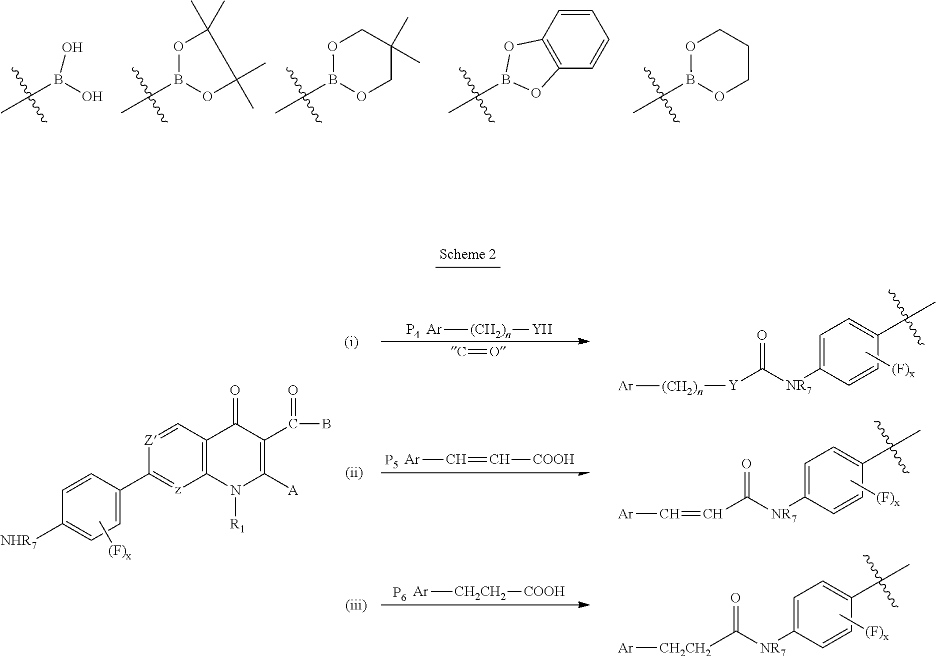

According to Scheme 2, P₃ reacts via —NHR₇ with:
P₄ of formula Ar—(CH₂)ₙ—YH in the presence of an agent for introducing the “C═O” unit (stage (i)). This agent may, for example, be phosgene, triphosgene or N,N′-disuccinimidyl carbonate (DSC). Advantageously, the reaction is carried out in the presence of triphosgene. It is also preferably carried out in the presence of a base such as, for example, triethylamine and at a temperature of between −5° C. and ambient temperature, in an ethereal solvent such as THF; or
P₅ of formula Ar—CH═CH—COOH (stage (ii)) or P₆ of formula Ar—CH₂CH₂—COOH (stage (iii)) by means of an amidation reaction advantageously carried out in the presence of an acid activator (or coupling agent), the function of which is to promote the formation of the amide bond; as an example of an acid activator, mention may be made of benzotriazol-1-yloxytris(dimethylamino)phosphonium hexafluorophosphate (or BOP, CAS No.: 56602-33-6, see B. Castro., Dormoy, J. R. Tetrahedron Letter 1975, 16, 1219). For other acid activators, see “Principles of peptide synthesis” 1993, 2^{nd} Ed, Bodanszky, Springer Laboratory.

When Y represents NH, an alternative consists in reacting P₅′ of formula Ar—(CH₂)ₙ—NH—C(═O)O-Ph-p-NO₂ with P₃ under the conditions of example 7.6. An example of P₅′ is pyridin-3-ylmethylcarbamic acid 4-nitrophenyl ester.

### Preparation of Intermediate Compounds

### Preparation of P₁

P₁ with L=—(CH₂)ₙY— is obtained according to Scheme 3, by reacting P₄ and P₇ in the presence of an agent for introducing the “C═O” unit (for example, phosgene, triphosgene or DSC) by means of a reaction similar to that of the previous stage (ii):

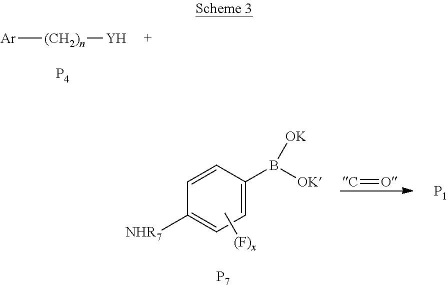

Example 1.5 illustrates the preparation of P₁ when P₄ represents 3-(aminomethyl)pyridine. An example of P₇ that is advantageously used in Scheme 3 and in the following schemes is 4-(4,4,5,5-tetramethyl[1,3,2]dioxaborolan-2-yl)phenylamine (commercial product).

P₁ with L=—CH═CH— or —CH₂CH₂— is obtained by means of an amidation reaction between P₇ and respectively P₅ or P₆, similar to that of stage (iii) or (iv) of Scheme 2 above. Example 2.1 illustrates the preparation of a P₁ when P₅ represents 3-(3-pyridyl)acrylic acid.

P₁ with L=—CH₂CH[NHC(═O)O(C₁-C₄)alkyl]- is obtained likewise by means of an amidation reaction between P₇ and the P′₆ of formula: Ar—CH₂CH[NHC(═O)O(C₁-C₄)alkyl]-COOH. For example, for compound no. 16, use is made, for P′₆, of the following commercial product:

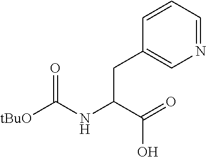

Preparation of P₂ with A=—NH₂ and B=—NR₄R₅

### When Z=N and Z′=CH

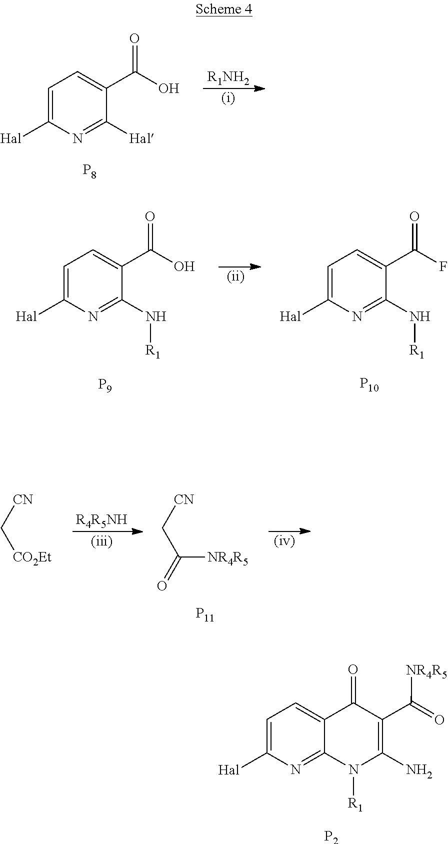

P₂ is obtained starting from a 2,6-dihalonicotinic acid P₈ in which Hal and Hal′ both represent a halogen atom; for example, it may be 2,6-dichloronicotinic acid (Hal=Hal′=Cl). Those skilled in the art may use the conditions given in example 1 as a reference and apply them to other R₁ and R₅ groups:

stage (i): the reaction with the amine R₁NH₂ can take place at ambient temperature, in a protic solvent such as an alcohol or water (cf. example 1.1);
stage (ii): conversion of the acid P₉ to acid fluoride P₁₀ using, for example, cyanuric fluoride and, optionally, a base such as pyridine. The reaction can take place at ambient temperature, in dichloromethane (DCM) (cf. example 1.2). For further details on this reaction, reference may be made to Synthesis 1973, 487, “Synthetic Methods and Reactions; IV.1 Fluorination of Carboxylic Acids with Cyanuric Fluoride”;
stage (iii): this reaction between ethyl cyanoacetate and the amine (R₄R₅NH₂ or R₅NH₂) in excess, which makes it possible to obtain an N-alkylcyanoacetamide, can take place in a polar solvent such as THF or ethanol, at a temperature ranging from −50° C. to ambient temperature (cf. example 1.3);
stage (iv): P₁₀ and P₁₁ are brought into contact in the presence of at least two equivalents of a strong base and the mixture is left to react at ambient temperature for a few hours. Another equivalent of strong base is then added to the reaction mixture and the intermediate compound formed cyclizes in situ at ambient temperature. The strong base may be NaH or else NaH combined with t-BuOK. A solution of P₁₀ is preferably added in small amounts to a solution of P₁₁ (cf. example 1.4).

### When Z=Z′=CH

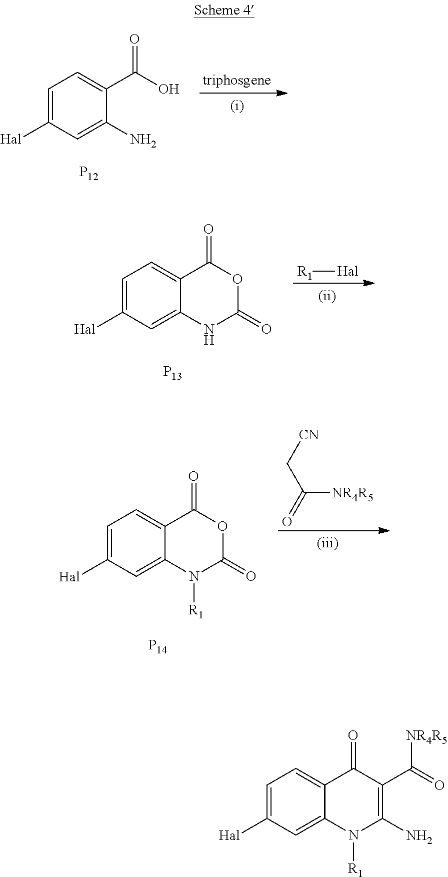

P₂ is obtained starting from a 2-amino-4-halobenzoic acid P₁₂, for example, it may be 2-amino-4-bromobenzoic acid (Hal=Br). Those skilled in the art may use the conditions given in example 7 as a reference and apply them to other R₁ and R₅ groups.

stage (i): reaction of P₁₂ with triphosgene so as to obtain P₁₃. The reaction may be carried out in dioxane reflux (cf. example 7.1);
stage (ii): reaction of P₁₃ with a halide R₁Hal in the presence of a strong base, in the presence of NaH, in a polar solvent, for example DMF (cf. example 7.2). The halide is more particularly an iodide (for example, ethyl iodide);
stage (iii): reaction between P₁₁ and P₁₄ in the presence of a strong base, for example NaH, in a polar solvent, for example DMF (cf. example 7.3).

### When Z=Z′=N

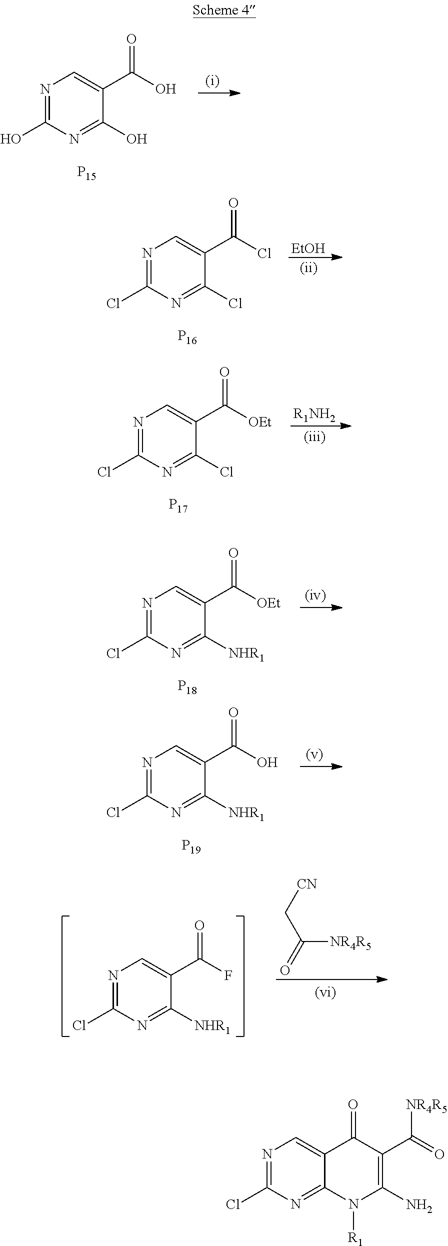

P₂ is obtained starting from the 2,4-dihydroxypyrimidine-5-carboxylic acid P₁₅. Those skilled in the art may use the conditions given in example 9 as a reference and apply them to other R₁ and R₅ groups:

stage (i): preparation of the acid chloride P₁₆. A POCl₃/PCl₅ mixture may be used as acylating agent (cf. example 9.1);
stage (ii): preparation of the ester P₁₇ by bringing the acid chloride into contact with ethanol (cf. example 9.2);
stage (iii): reaction of P₁₆ with the amine R₁NH₂. The reaction may take place in a polar solvent such as THF (cf. example 9.3);
stage (iv): conversion of the ester function —COOEt to an acid function —COOH. It is possible to use a base such as lithium hydroxide in water and then to acidify (cf. example 9.4);
stage (v): preparation of the acid fluoride. Cyanuric fluoride in DCM may be used (cf. example 9.5);
stage (vi): cyclization reaction between P₁₁ and the acid fluoride according to the same method as that described in stage (iv) of Scheme 4 (cf. also example 9.6).
Preparation of P₂ with A=—NH₂ and B=—OR₆

This compound is obtained according to Scheme 5 by reacting P₂₀, in which R represents a (C₁-C₄)alkyl group, with NH₃. More particularly, R₆=ethyl and/or R=methyl.

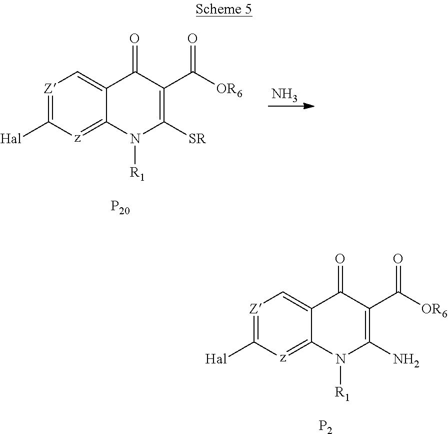

The reaction may be carried out in the presence of a concentrated aqueous ammonia solution, at ambient temperature (cf. example 3.5).

### Preparation of P₃

P₃ with R₇=H is obtained according to Scheme 6 by Suzuki coupling (under the conditions previously described) between P₇ and P₂:

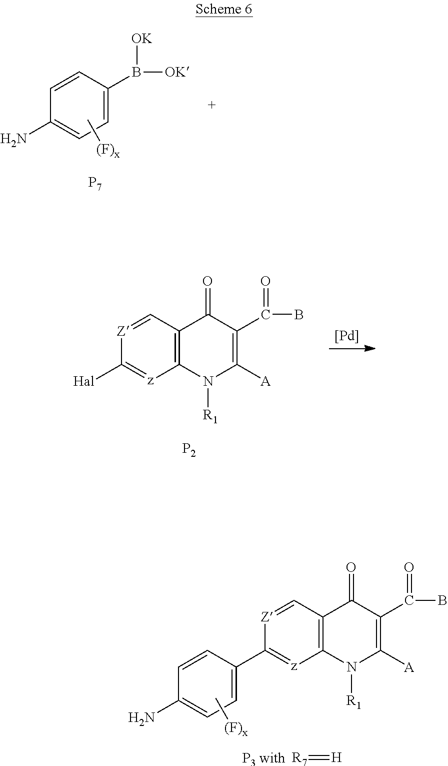

When R₇ represents a (C₁-C₄)alkyl group, use is made of Scheme 6′ (variant of Scheme 6) in which P₇ is replaced with P₇′. P₇′ is obtained by introducing the R₇ group by alkylation of the amine function of P₇ protected beforehand with the protective group PG (PG may, for example, be the tert-butyloxycarbonyl, BOC, group).

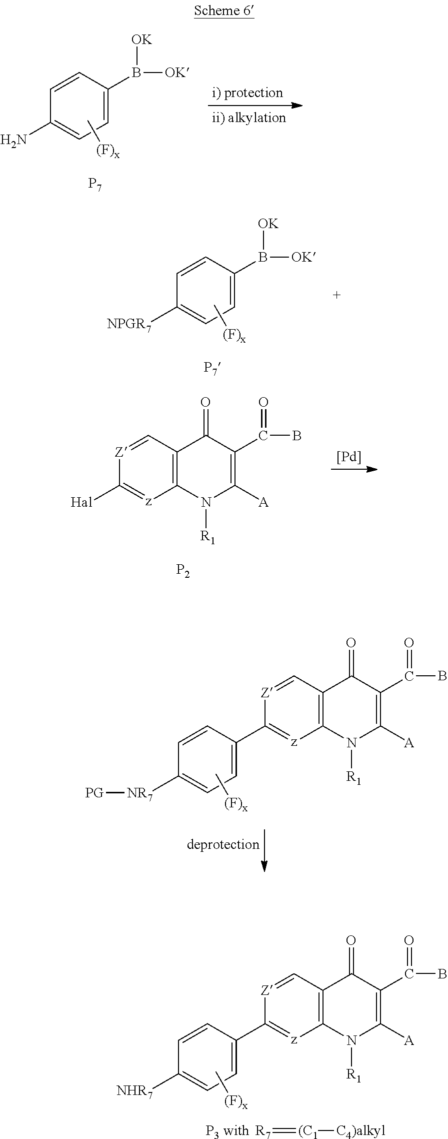

In the Particular Case when A and B Both Represent the —NH—(C₁-C₆)alkyl-Q-Group

In this case, P₃ is obtained according to Scheme 6″ by Suzuki coupling between P₇ and P₂₀ so as to give P₂₁, followed by reaction of P₂₁ with the compound of formula H₂N—(C₁-C₆)alkyl-Q′. Q′ has the same definition as Q, except when Q represents an —NH₂ group, in which case Q′ represents the —NH-PG group in which PG represents a protective group for the amine function (for example, BOC) and the coupling is then followed by a deprotection stage which makes it possible to form the —NH₂ function.

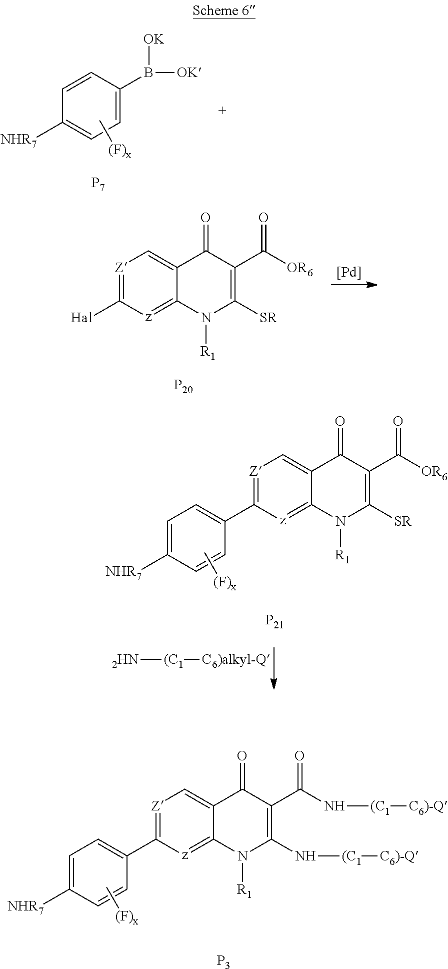

The reaction between P₂₁ and H₂N—(C₁-C₆)alkyl-Q′ is preferably carried out at a temperature above 100° C. (cf. example 8.7 or 4.2) and preferably for a long period of time (>10 h).

### Preparation of P₂₀

P₂₀ is prepared starting from P′₈ according to Scheme 7:

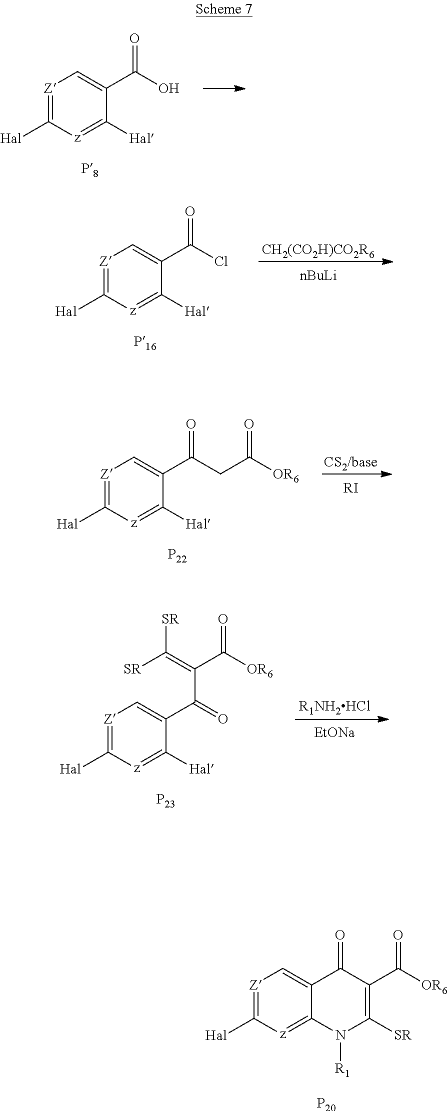

stage (i): the acid is converted to acid chloride. Thionyl chloride or oxalyl chloride (COCl)₂ may, for example, be used (cf. example 3.1 or 8.1). When Z=Z′=N, 2,4-dihydroxypyrimidine-5-carboxylic acid is used in place of P′₈ in order to obtain P′₁₆ (see example 9.1);
stage (ii): the acid chloride then reacts with the alkyl monomalonate CH₂(CO₂H)COOR₆ which, beforehand, will have been brought into contact with n-BuLi so as to give a β-keto ester P₂₂ (cf. example 3.2 or 8.2);
stage (iii): the β-keto ester P₂₂ is brought into contact with a base such as K₂CO₃ and with CS₂ in a solvent, for example THF, and then an iodide RI is added (cf. example 3.3 or 8.3) so as to obtain P₂₃;
Stage (iv): P₂₀ is obtained by reacting P₂₃ with the amine R₁NH₂ (free base or hydrochloride) in the presence of a base, such as, for example, NaHCO₃ or K₂CO₃ (cf. example 3.4 or 8.4). This stage (iv) can be carried out in a single step (cf. example 3.4) or with an intermediate compound being isolated (cf. example 8.4).

### Preparation of P₇

The compounds P₇ for which K and K′ form the following group:

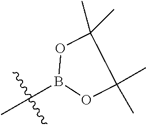

are commercially available or can be prepared according to the coupling reaction between an optionally fluorinated bromoaniline and bis(pinacolato)diboron which is described in scheme 2 on pages 150-151 of WO 2007/064931; for example, in the case of the fluorinated compounds: 3-F (4-amino-3-fluorophenylboronic acid pinacol ester, CAS No. 819058-34-9, the company Boron Molecular Inc, PO Box 12592, Research Triangle Park, N.C. 27709); 2-F (4-amino-2-fluorophenylboronic acid pinacol ester, CAS No. 819057-45-9, Boron Molecular, described on page 185 of WO 2007/064931); 2-F, 5-F (CAS No. 939807-75-7, compound described on page 184 of WO 2007/064931); 3-F, 5-F (CAS No. 939968-08-8, described on page 182 of WO 2007/064931). The fluorinated compounds P₇ for which K and K′ represent a hydrogen atom can be prepared from fluorinated bromoaniline by means of the reactions described in Tetrahedron Letters 2003, 44, 7719-7722.

### Preparation of the Amine R₄R₅NH₂ or R₅NH₂

The amines are commercial products or products already described in published documents; for example:
1-(2-aminoethyl)piperidine: CAS No. 27578-60-5, described in Justus Liebigs Annalen der Chemie 1950, 566, 210-44, sold by ACROS;
1-(2-aminoethyl)-4-piperidinol: CAS No. 129999-60-6, described in J. Med. Chem. 2005, 48(21), 6690-6695 and also on page 17 of WO 2005/061453 (ref. example 10);
pyrrolidineethanamine: CAS No. 7154-73-6, described in Anales de Quimica 1974, 70(9-10), 733-737, sold by International Laboratory Ltd, 1067 Sneath Ln, San Bruno, Calif. 94066, USA;
1-piperazineethanamine: CAS No. 140-31-8, described in EP 151232;
4-Morpholineethanamine: CAS No. 2038-03-1, described in Khimiya Prirodnykh Soedinenii, (5), 707-12; 1989;
4,4-difluoro-1-piperidineethanamine: CAS No. 605659-03-8, described on page 46 of US 2006058308;
4-piperidineethanamine: CAS No. 76025-62-2, described in J. Org. Chem. 1961, 26, 3805-8;
2-tetrahydrofurylmethanamine: CAS No. 4795-29-3, described in Ind. Alim. et Agric. 1987, 104(1-2), 45-51;
2-amino-2-(hydroxymethyl)propane-1,3-diol: CAS No. 77-86-1, described in EP 287419 (examples 1-3).

A general method for obtaining amines is also described on scheme 8 and is based on scheme 3 of Bioorg. Med. Chem. 2007, 15, 365-373 or on scheme 2 of Bioorg. Med. Chem. Lett. 2008, 18, 1378-1381:

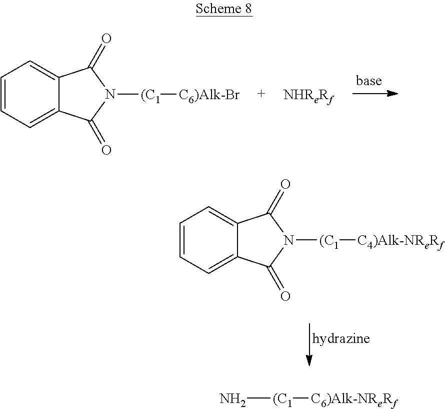

Another general method described on scheme 9 is based on FIG. 2 of Bioorg. Med. Chem. Lett. 2006, 16, 1938-1940:

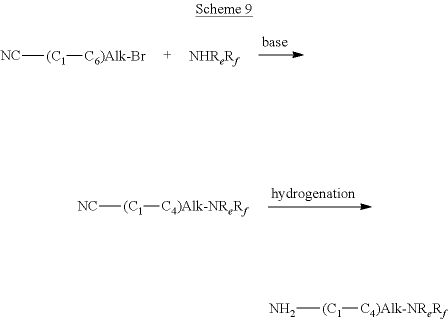

### Compounds P₄

These products are either commercially available or are described in publications or patent applications; for example:
3-pyridinemethanamine: CAS No. 3731-52-0, described in Chem.-Eur. J. 2008, 14(31), 9491-9494;
2-amino-5-aminomethylpyridine: CAS No. 156973-09-0, described in US 2005137395 (compound 117B, page 70);
3-(aminomethyl)aniline: CAS No. 4403-70-7, commercial product, described in Synthesis 2001, 1, 81-84 ;
3-pyridineethanamine: CAS No. 20173-24-4, described in Chem. Ber. 1947, 80, 505-9;
5-thiazolemethanamine: CAS No. 161805-76-1, commercial product.

### Compounds P₅ or P₆ (Ar—CH═CH—COOH or Ar—CH₂CH₂—COOH)

These products are either commercially available or are described in publications or patent applications, for example:
3-(3-pyridyl)-2-propenoic acid: CAS No. 1126-74-5, commercial product;
3-piperidinepropanoic acid: CAS No. 3724-19-4, described in Chem. Ber. 1947, 80, 505-9;
3-(6-aminopyridin-3-yl)acrylic acid: CAS No. 234098-57-8, described in WO 00/32590 (example 36);
6-amino-3-pyridinepropanoic acid: CAS No. 446263-96-3, described in EP 1394159 (preparation 6, page 27);
3-(3-aminophenyl)-2-propenoic acid: CAS No. 1664-56-8, commercial product;
3-aminobenzenepropanoic acid: CAS 1664-54-6, commercial product;
3-(5-thiazolyl)-2-propenoic acid: CAS No. 355009-32-4, commercial product;
5-thiazolepropanoic acid: CAS No. 933724-95-9, available from CHEMSTEP, 20, ay.V.Hugo, 33560 France.

In the above schemes, the starting compounds and the reactants, when the method for preparing them is not described, are commercially available or described in the literature, or else can be prepared according to methods which are described therein or which are known to those skilled in the art. Those skilled in the art may use the operating conditions given in the examples, which are described hereinafter, as a reference.

In the above schemes, it may be necessary to use, in at least one of the stages, a protective group in order to protect a chemical function (cf., for example, scheme 6′). For example, when R_{c} and R_{d} both represent a hydrogen atom, the reaction of scheme 6′ is carried out with the compound of formula H₂N—(C₁-C₆)alkyl-NH-PG where PG denotes a protective group for the amine function (it is advantageously the tert-butyloxycarbonyl, BOC, group). The —NH₂ function is subsequently obtained by means of a deprotection stage which makes it possible to go from —NH—PG to —NH₂, for example by means of a treatment in an acidic medium. Thus, for compound No. 50, the compound H₂N—(CH₂)₆—NH—BOC is used.

### Obtaining the Salts

The salts are obtained by bringing the acid and the compound, in the form of its base, into contact. They can also be obtained during a stage for deprotection in an acidic medium.

According to a 3^{rd} aspect, the invention relates to a pharmaceutical composition comprising a compound as defined above, in combination with a pharmaceutically acceptable excipient. The excipient is chosen, according to the pharmaceutical form and the method of administration desired, from the usual excipients which are known to those skilled in the art. The method of administration may be oral or intravenous administration.

According to a 4^{th} aspect, a subject of the invention is a medicament which comprises a compound as defined above, and also the use of a compound as defined above, for the manufacture of a medicament. It may be of use for treating a pathological condition, in particular cancer. This medicament may find use in the treatment or prevention of diseases caused or exacerbated by the proliferation of cells, and in particular of tumour cells.

The medicament may be administered in combination with one (or more) other anticancer agent(s), in particular chosen from:
chemotherapy agents, such as alkylating agents, platinum derivatives, antibiotics, antimicrotubule agents, taxoids, anthracyclines, topoisomerase type I and II inhibitors, fluoropyrimidines, cytidine analogues, adenosine analogues, enzymes, and also oestrogenic and androgenic hormones;
antivascular agents;
anti-angiogenic agents.

It is also possible to combine a radiation treatment. This treatment may be administered simultaneously, separately or else sequentially. The treatment will be adapted by the practitioner as a function of the disease and of the tumour to be treated.

According to a 5^{th} aspect, the invention also relates to a method for treating the pathologies indicated above, which comprises the administration, to a patient, of an effective dose of a compound according to the invention or a pharmaceutically acceptable salt or hydrate or solvate thereof.

### EXAMPLES

The following examples illustrate the preparation of some compounds in accordance with the invention. The numbers of the compounds exemplified refer back to those given in table I hereinafter, which illustrates the chemical structures and the physical properties of some compounds according to the invention.

### Mass Spectrometry Conditions

Conditions A to C: The compounds were analyzed by HPLC-UV-MS coupling (liquid chromatography, ultraviolet (UV) detection and mass detection). The apparatus used is composed of an Agilent chromatographic system equipped with an Agilent diode array detector and with a Waters ZQ single quadripole mass spectrometer or a Waters Quattro-Micro triple quadripole mass spectrometer. The liquid phase chromatography/mass spectrometer (LC/MS) spectra were recorded in positive electrospray (ESI) mode, in order to observe the ions resulting from the protonation of compounds analyzed (MH⁺) or from the formation of adducts with other cations, such as Na⁺, K⁺, etc. The ionization parameters are as follows: cone voltage: 20 V; capillary voltage: 3 kV; source temperature: 120° C.; desolvation temperature: 450° C.; desolvation gas: N₂ at 450 l/h.

Conditions D: The compounds are analyzed by HPLC-UV-MS coupling (liquid chromatography—UV detection and mass detection). The apparatus used is composed of a Gilson chromatographic system equipped with an Agilent diode array detector and with a Thermo Finnigan AQA single quadripole mass spectrometer. The mass spectra are recorded in positive electrospray (ESI) mode at a cone voltage of 20 kV, in order to observe the ions derived from the protonation of compounds analyzed (MH⁺) or from the formation of adducts with other cations, such as Na⁺, K⁺, etc.

The conditions of the LCMS are chosen from one of the following methods:

| Conditions | [symbol] A [symbol] | | [symbol] B [symbol] | | [symbol] C [symbol] | | [symbol] D [symbol] | |
|---|---|---|---|---|---|---|---|---|
| column type | symmetry C18 | | symmetry C18 | | XTerra MS C18 | | Macherey-Nagel | |
| | (50 × 2.1 mm; | | (50 × 2.1 mm; | | (50 × 2.1 mm; | | Nucleodur C18 | |
| | 3.5 μm) | | 3.5 μm) | | 3.5 μm) | | (150 × 4.6 mm; | |
| | | | | | | | 5.0 μm) | |
| eluant A | H₂O + 0.005% | | H₂O + 0.005% | | 10 mM | | H₂O + 0.1% | |
| | TFA at | | TFA at | | AcONH₄ at | | HCOOH at | |
| | approximately | | approximately | | pH 7 | | approximately | |
| | pH 3.1 | | pH 3.1 | | | | pH 2.0 | |
| eluant B | CH₃CN + | | CH₃CN + | | CH₃CN | | CH₃CN + 0.1% | |
| | 0.005% TFA | | 0.005% TFA | | | | HCOOH | |
| gradient | 100:0 | (0 min) to | 100:0 | (0 min) to | 100:0 | (0 min) to | min | (% B): |
| | 10:90 | (10 min) to | 10:90 | (20 min) to | 10:90 | (10 min) to | 0 | (5%); |
| | 100:0 | (15 min) | 100:0 | (30 min) | 100:0 | (20 min) | 11.5 | (5%); |
| | | | | | | | 9.5 | (95%); |
| | | | | | | | 15.5 | (95%); |
| | | | | | | | 16.3 | (5%) |
| column | 30° | C. | 30° | C. | 30° | C. | 25° | C. |
| temperature | | | | | | | | |
| flow rate | 0.4 | ml/min | 0.4 | ml/min | 0.4 | ml/min | 1.0 | ml/min |

| | | | | |
|---|---|---|---|---|
| detection | λ = 220 nm | λ = 220 nm | λ = 220 nm | λ = 245-345 nm |
| wavelength | | | | |
| lambda λ | | | | |

TFA: trifluoroacetic acid
LCMS (A): tr = 6.52 min signifies LCMS under conditions A with retention time of 6.52 minutes

### Example 1

### 2-Amino-1-ethyl-4-oxo-7-[4-(3-pyridin-3-ylmethylureido)phenyl]-1,4-dihydro[1,8]naphthyridine-3-carboxylic Acid Methylamide (No. 1)

### 1.1: 2-(Aminoethyl)-6-chloronicotinic Acid

A solution of 18.0 g (84.4 mmol) of 2,6-dichloronicotinic acid in 180 ml of a solution of ethylamine at 70% in water is stirred at ambient temperature for 72 hours. The excess amine is then evaporated off under reduced pressure, and a 10% aqueous solution of acetic acid is then added until the product precipitates. The beige solid is spin-filter-dried, rinsed with cold water and oven-dried. 10.5 g of the expected product are obtained. Mp (melting point)=158-160° C. Yield=62%.

### 1.2: 2-(Aminoethyl)-6-chloronicotinic Acid Fluoride

2 ml (24.8 mmol) of pyridine and 4.2 ml (49.8 mmol) of cyanuric fluoride are added to a suspension of 5.0 g (24.8 mmol) of 2-(aminoethyl)-6-chloronicotinic acid in 125 ml of dichloromethane. The mixture is stirred for 3 h at ambient temperature and then filtered. The solid is rinsed with 50 ml of dichloromethane and the filtrate is washed twice with 60 ml of ice-cold water. The organic phase is dried over Na₂SO₄ and the solvent is evaporated off under reduced pressure. 5.01 g of product are obtained in the form of an orange solid. Yield=99%.

### 1.3: N-methylcyanoacetamide

12.28 ml (128.44 mmol) of ethyl chloroformate are added, dropwise, to a solution, cooled to −30° C., of 10.0 g (116.38 mmol) of cyanoacetic acid at 99% and 16.3 ml (116.9 mmol) of triethylamine in 100 ml of anhydrous THF, and then the mixture is stirred at −30° C. for one and a half hours. 300 ml of methanol saturated with methylamine gas are subsequently added, dropwise, and then the mixture is stirred at ambient temperature overnight. The solvents are evaporated off under reduced pressure and the product is purified by silica gel chromatography, elution being carried out with a dichloromethane:methanol (95:5) mixture. 10.0 g of product are obtained in the form of a beige solid. Mp=99° C. Yield=87%.

### 1.3: N-methylcyanoacetamide

### Variant

A stream of methylamine (gas) is passed through a solution of 30.0 g (265.2 mmol) of ethyl cyanoacetate in 220 ml of THF, for 2 h, while maintaining the temperature below 25° C. The THF is evaporated off and 25.86 g of white solid are obtained. Yield=99.4%; Mp=100-102° C.

### 1.4: 2-amino-7-chloro-1-ethyl-N-methyl-4-oxo-1,4-dihydro-1,8-naphthyridine-3-carboxamide

0.394 g (9.95 mmol) of sodium hydride at 60% in mineral oil is added, in small amounts, to a solution, cooled to 0-5° C., of 0.483 g (4.93 mmol) of N-methylcyanoacetamide in 7 ml of anhydrous DMF. The stirring is continued at this temperature for ten minutes and then a solution of 1.0 g (4.93 mmol) of 2-(aminoethyl)-6-chloronicotinic acid fluoride in 5 ml of DMF is added. The medium is stirred overnight at ambient temperature and then a further 0.197 g (4.93 mmol) of sodium hydride at 60% is added in small amounts. The stirring is continued at this temperature for 10 minutes and then 0.56 ml (9.78 mmol) of acetic acid is added. 60 ml of water are then added and the solid is spin-filter-dried, rinsed with water, and then oven-dried. 1.30 g of the expected product are obtained. Mp=283-284° C. Yield=94%.

### 1.5: 1-Pyridinyl-3-ylmethyl-3-[4-(4,4,5,5-tetramethyl[1,3,2]dioxaborolan-2-yl)phenyl]urea

57.2 ml (410.8 mM) of triethylamine are introduced, dropwise, into a mixture of 15 g (68.47 mmol) of 4-(4,4,5,5-tetramethyl[1,3,2]dioxaborolan-2-yl)phenylamine and 12.19 g (41.08 mM) of triphosgene in 1.5 l of THF, cooled by means of an ice/water bath to a temperature of between 0° C. and 5° C. After stirring for 1 h at a temperature of between 0° C. and 5° C., 8.29 g (76.68 mM) of 3-(aminomethyl)pyridine are added to the reaction medium. The reaction medium is stirred for 20 h while allowing the temperature to increase to ambient temperature. The THF is evaporated off. The residue is taken up in water and then extracted with ethyl acetate. The organic phase is then dried over Na₂SO₄, filtered and evaporated. The residue is recrystallized from a minimum amount of ethyl acetate. 13 g (yield=53.8%) of white solid are obtained, composed of 89% of expected compound and 11% of corresponding boronic acid (LC/MS (A); M+ 353 and 271 tr=6.25 and 3.65 min).

### 1.6: 2-Amino-1-ethyl-4-oxo-7-[4-(3-pyridin-3-ylmethylureido)phenyl]-1,4-dihydro[1,8]naphthyridine-3-carboxylic Acid Methylamide

0.722 g (2.57 mmol) of compound obtained in stage 1.4, 1.11 g (2.83 mmol) of compound obtained in stage 1.5, 38 ml of DME, 7.5 ml of ethanol and 17.7 ml of a saturated solution of NaHCO₃ are placed in a three-necked flask. The mixture is degassed with argon and then 0.297 g (0.26 mmol) of Pd(PPh₃)₄ is added. The mixture is heated at 100° C. for 2 h. After a return to ambient temperature, the precipitate is filtered off and washed with DME and water. The resulting product is oven-dried over P₂O₅. 0.896 g of product is obtained. Yield=74%. LCMS (A) M=472 tr=5.74 min.

### 1.7: 2-Amino-1-ethyl-4-oxo-7-[4-(3-pyridin-3-ylmethylureido)phenyl]-1,4-dihydro[1,8]naphthyridine-3-carboxylic Acid Methylamide Hydrochloride

0.790 g of the compound obtained in stage 1.6 is dissolved in DMF and a solution of hydrochloric ether is added and then the mixture is evaporated to dryness. The residue is taken up with methanol and filtered. The resulting product is oven-dried. 0.637 g of the product is obtained Yield=74.8%. LCMS (A) M=472 tr=5.74 min. ¹H NMR (DMSO-d6, 200 MHz): 1.31 (t, 3); 2.8 (s, 3); 4.51 (d, 2); 4.59 (quad, 2); 7.26 (t, 1); 7.59 (d, 2); 7.88 (d, 1); 7.99 (dd, 1); 8.12 (d, 2); 8.46 (d, 2); 8.8 (d, 1); 8.85 (s, 1); 11.13 (s, 1); 11.69 (sl, 1). IC₅₀ (HCT116)=0.2-0.5 nM.

### Example 2

### 2-Amino-1-ethyl-4-oxo-7-[4-((E)-3-pyridin-3-ylacryloylamino)phenyl]-1,4-dihydro[1,8]naphthyridine-3-carboxylic Acid Methylamide (No. 7)

### 2.1: (E)-3-Pyridin-3-yl-N-[4-(4,4,5,5-tetramethyl[1,3,2]dioxaborolan-2-yl)phenyl]acrylamide

0.746 g (5 mmol) of 3-(3-pyridyl)acrylic acid and 1.095 g (5 mmol) of 4-(4,4,5,5-tetramethyl[1,3,2]dioxaborolan-2-yl)phenylamine in suspension in 60 ml of THF are mixed, and 0.7 ml of triethylamine and 2.06 g (5 mmol) of BOP are added. The mixture is refluxed for 18 h. The THF is evaporated off and the residue is taken up with ethyl acetate. The organic phase is washed with water and a saturated solution of NaCl. The resulting product is dried over Na₂SO₄, filtered and evaporated. The residue is purified by silica column chromatography with a 95:5 mixture of CH₂Cl₂:MeOH. 1.0 g of product is obtained. Yield=57%. LCMS (A) M=351; tr=8.05 min.

### 2.2: 2-Amino-1-ethyl-4-oxo-7-[4-((E)-3-pyridin-3-ylacryloylamino)phenyl]-1,4-dihydro[1,8]naphthyridine-3-carboxylic Acid Methylamide

The coupling conditions of stage 1.6 are repeated, using the compound of stage 2.1 above and that of stage 1.4; the expected product is obtained with a yield of 65%. LCMS (A) M=469 tr=7.05 min. ¹H NMR (DMSO-d6, 250 MHz): 1.31 (t, 3); 2.79 (d, 3); 4.6 (quad, 2); 6.96 (d, 1); 7.46 (m, 1); 7.67 (d, 1); 7.8-7.98 (unresolved peak, 3); 8.05 (d, 1); 8.22 (d, 2); 8.49 (d, 1); 8.59 (d, 1); 8.84 (s, 1); 10.53 (s, 1); 11.12 (m, 1); 11.73 (sl, 1). IC₅₀ (HCT116)=2 nM

### Example 3

### 2-Amino-1-ethyl-4-oxo-7-[4-(3-pyridin-3-ylmethylureido)phenyl]-1,4-dihydro[1,8]naphthyridine-3-carboxylic Acid Ethyl Ester (No. 10)

### 3.1: 2,6-dichloronicotinic Acid Chloride

58 ml (0.796 mmol) of thionyl chloride are added, dropwise, to a suspension of 30.58 g (0.159 mmol) of 2,6-dichloronicotinic acid in 200 ml of chloroform and 0.1 ml of DMF, and then the mixture is refluxed for 17 hours. The mixture is allowed to return to ambient temperature and then the solvent is evaporated off under reduced pressure. The residue obtained is taken up in toluene and evaporated to dryness. 33.53 g of an amber liquid are obtained. The yield is quantitative.

### 3.2: Ethyl 3-(2,6-dichloro-3-pyridinyl)-3-oxopropanoate

250 ml (0.625 mol) of a 2.5 M solution of N-butyllithium in hexane are added, dropwise, to a cooled solution of 41.3 g (0.313 mol) of ethyl monomalonate in 860 ml of THF under argon. 10 minutes after the addition has been completed, the reaction medium is cooled to −50° C. and a solution of 21.84 g (0.104 mol) of 2,6-dichloronicotinic acid chloride in 160 ml of THF is added dropwise. The mixture is allowed to return to ambient temperature and stirring is continued for 1 h. 625 ml (0.625 mol) of a 1 N aqueous solution of hydrochloric acid are then added and the product is extracted several times with ether. The organic phase is dried over MgSO₄ and the solvent is evaporated off under reduced pressure. The product is purified by silica gel chromatography, elution being carried out with a dichloromethane:n-heptane (9:1) mixture. 23.65 g of product are obtained in the form of an oil. Yield=87%. LCMS (C) M=261 tr=8.04 min.

### 3.3: Ethyl 2-[(2,6-dichloro-3-pyridinyl)carbonyl]-3,3-bis(methylthio)acrylate

A solution of 2.82 ml (46.9 mmol) of CS₂ in 0.5 ml of DMF is introduced, dropwise, into a mixture, cooled to −10° C. under argon, of 10.25 g (39.11 mmol) of the β-keto ester (compound obtained in stage 3.2) and 16.21 g (117.29 mmol) of K₂CO₃ in 78 ml of DMF. After two minutes, a solution of 7.30 ml (117.28 mmol) of methyl iodide in 4.5 ml of DMF is rapidly added. The reaction medium is stirred for 2 h at 0-5° C. and then the temperature is allowed to return to ambient temperature for 1 hour. The solvent is evaporated off under reduced pressure and 250 ml of water are poured onto the residue. The product is extracted several times with ethyl acetate and the combined organic phases are washed with water. The organic phase is dried over MgSO₄ and the solvent is evaporated off under reduced pressure. The product is purified by silica gel chromatography, elution being carried out with dichloromethane. 11.28 g of product are obtained in the form of an oil. Yield=79%. LCMS (A) M=365, tr=9.70 min.

### 3.4: Ethyl 7-chloro-1-ethyl-2-(methylthio)-4-oxo-1,4-dihydro-1,8-naphthyridine-3-carboxylate

A solution of 2.59 g (31.76 mmol) of ethylamine hydrochloride and 2.25 g (33.06 mmol) of sodium ethoxide at 96% in 20 ml of absolute ethanol is added to a solution, cooled to 0-5° C., of 11.12 g (30.36 mmol) of the compound obtained in stage 3.3, in 95 ml of dichloromethane, and the mixture is stirred for 22 h at normal temperature. The reaction medium is degassed and the solvents are then evaporated off under reduced pressure. The residue obtained is taken up in 100 ml of DMF and then 3.06 g (36.40 mmol) of NaHCO₃ are added and the mixture is heated at 80° C. for 5 h. The solvent is evaporated off under reduced pressure and the residue is taken up in dichloromethane. The solution is then washed twice with water and then with a saturated aqueous solution of sodium chloride. The organic phase is dried over Na₂SO₄ and the solvent is evaporated off under reduced pressure. The product is purified by silica gel chromatography, elution being carried out with a mixture of n-heptane:ethyl acetate (2:1, then 3:2). 7.09 g of product are obtained in the form of a pale yellow syrup. Yield=72%. LCMS (A) M=326 tr=8.77 min.

### 3.5: Ethyl 2-amino-7-chloro-1-ethyl-4-oxo-1,4-dihydro-1,8-naphthyridine-3-carboxylate

8 ml (56.9 mmol) of a concentrated aqueous ammonia solution are added to a solution of 1.86 g (5.69 mmol) of the compound obtained at the end of stage 3.4, in 27 ml of acetonitrile. The vessel is hermetically sealed and the reaction medium is stirred at ambient temperature for 72 h. The solvents are evaporated off under reduced pressure and the residue obtained is coevaporated twice with toluene. The residue is taken up in 20 ml of ice-cold diethyl ether, the mixture is stirred, and then the solid obtained is spin-filter-dried, rinsed with cold ether and dried under vacuum. 1.30 g of an off-white solid are obtained. Mp=252° C. Yield=77%.

### 3.6: 2-Amino-1-ethyl-4-oxo-7-[4-(3-pyridin-3-ylmethylureido)phenyl]1,4-dihydro[1,8]naphthyridine-3-carboxylic Acid Ethyl Ester

In a three-necked flask, 2.3 g (7.78 mmol) of compound obtained in stage 3.5 and 3.02 g (8.56 mmol) of 1-pyridinyl-3-ylmethyl-3-[4-(4,4,5,5-tetramethyl[1,3,2]dioxaborolan-2-yl)phenyl]urea are placed in 120 ml of DME, 24 ml of ethanol and 55 ml of a saturated solution of NaHCO₃. The mixture is degassed with argon and then 0.899 g (0.78 mmol) of Pd(PPh₃)₄ is added. The mixture is heated at 110° C. for 3 h. After a return to ambient temperature, the mixture is diluted with water and the resulting mixture is filtered, and the precipitate is washed with water and ethyl acetate. The resulting product is vacuum-dried over P₂O₅ and recrystallized from i-PrOH. 3.64 g are obtained. Yield=96%. LCMS (A): M=486 tr=5.68 min. ¹H NMR (DMSO-d6 250 MHz): 1.18-1.36 (unresolved peak, 6); 4.2 (quad, 2); 4.33 (d, 2); 4.57 (quad, 2); 6.82 (t, 1); 7.4 (dd, 1); 7.58 (d, 2); 7.72 (d, 1); 7.85 (d, 1); 8.1 (d, 2); 8.37 (d, 1); 8.45 (d, 1); 8.54 (s, 1); 8.69 (s, 2); 8.93 (s, 1). IC₅₀ (HCT116)<1 nM.

### Example 4

### 1-Ethyl-2-[2-(4-methyl piperazin-1-yl)ethylamino]-4-oxo-7-[4-(3-pyridin-3-ylmethylureido)phenyl]-1,4-dihydro[1,8]naphthyridine-3-carboxylic Acid [2-(4-methylpiperazin-1-yl)ethyl]amide (No. 46)

### 4.1: 7-(4-Aminophenyl)-1-ethyl-2-methylsulfanyl-4-oxo-1,4-dihydro[1,8]naphthyridine-3-carboxylic Acid Ethyl Ester

6.24 g (19.09 mmol) of the compound obtained in stage 3.4, 4.6 g (21 mmol) of 4-(4,4,5,5-tetramethyl[1,3,2]dioxaborolan-2-yl)phenylamine, 280 ml of DME, 50 ml of ethanol and 130 ml of a saturated solution of NaHCO₃ are placed in a three-necked flask. The mixture is degassed with argon and 2.20 g (1.91 mmol) of Pd(PPh₃)₄ are added. The mixture is heated at 100° C. for 2 hours. The solvents are evaporated off and the residue is taken up with water and filtered and the precipitate is washed with water and vacuum-dried over P₂O₅. The resulting product is purified by silica chromatography, elution being carried out with a 95-5 mixture of CH₂Cl₂-MeOH. 4.7 g of product are obtained. Yield=64.2%. LCMS (A) M=383 tr=8.51 min.

### 4.2: 7-(4-Aminophenyl)-1-ethyl-2-[2-(4-methylpiperazin-1-yl)ethylamino]-4-oxo-1,4-dihydro[1,8]naphthyridine-3-carboxylic Acid [2-(4-methylpiperazin-1-yl)ethyl]amide

0.575 g (1.5 mmol) of compound obtained in stage 4.1 and 1.8 ml (18 mmol) of 2-(4-methylpiperazin-1-yl)ethylamine are placed in a screw-neck round-bottomed flask. The flask is hermetically sealed and the mixture is heated at 120° C. for 18 h. The residue is chromatographed on silica, elution being carried out with a 100-10-1 mixture of CH₂Cl₂-MeOH—NH₄OH. 0.685 g of product is obtained. Yield=79.4% LCMS (B) M=575 tr=6.36 min.

### 4.3: 1-Ethyl-2-[2-(4-methyl piperazin-1-yl)ethylamino]-4-oxo-7-[4-(3-pyridin-3-ylmethylureido)phenyl]-1,4-dihydro[1,8]naphthyridine-3-carboxylic Acid [2-(4-methylpiperazin-1-yl)ethyl]amide

0.657 g (1.14 mmol) of compound obtained in stage 4.2 is suspended in 40 ml of THF. 0.38 ml (2.74 mmol) of triethylamine, 0.167 g (1.37 mmol) of DMAP and 0.351 g (1.37 mmol) of DSC are added. The mixture is stirred at ambient temperature for 18 h. 0.148 g (1.37 mmol) of (3-aminomethyl)pyridine is added and stirring is continued for 20 hours. The resulting product is evaporated and the residue is purified by chromatography on silica with an 80-20-1 mixture of CH₂Cl₂-MeOH—NH₄OH. An oil is obtained, which solidifies when i-Pr₂O is added. The resulting product is filtered and dried under vacuum. 360 mg are obtained. Yield=44.4%. LCMS (A) M=709 tr=4.86 min. ¹H NMR (DMSO-d6 400 MHz): 1.38 (t, 3); 2.07 (s, 3); 2.16 (s, 3); 2.13-2.57 (unresolved peak, 20); 3.93 (quad, 2); 3.52 (quad, 2); 4.35 (d, 2); 4.56 (quad, 2); 6.86 (t, 1); 7.37 (dd, 1); 7.6 (d, 2); 7.73 (d, 1); 7.92 (d, 1); 8.14 (d, 2); 8.46 (d, 2); 8.55 (s, 1); 8.99 (s, 1); 11.14 (t, 1); 11.37 (t, 1). IC₅₀ (HCT116)=1.9 nM.

### Example 5

### 2-Amino-1-ethyl-4-oxo-7-[4-(3-pyridin-3-ylmethylureido)phenyl]-1,4-dihydro[1,8]naphthyridine-3-carboxylic Acid (No. 13)

1.338 g (2.75 mmol) of 2-amino-1-ethyl-4-oxo-7-[4-(3-pyridin-3-ylmethylureido)phenyl]-1,4-dihydro[1,8]naphthyridine-3-carboxylic acid ethyl ester and 0.33 g (8.25 mmol) of sodium hydroxide in 60 ml of dioxane+6 ml of water and 28 ml of ethanol are brought to reflux for 20 hours. After a return to ambient temperature, the mixture is filtered and the filtrate is evaporated off. The residue is taken up with water and a 1 N solution of HCl is added until dissolution is obtained; the pH is adjusted to 6-7 by adding sodium hydroxide, and the precipitate is filtered off. Drying is carried out under vacuum over P₂O₅. The precipitate is taken up with 35 ml of MeOH under hot conditions, the temperature is allowed to return to ambient temperature and filtration is carried out, and then the same operation is repeated with 25 ml of MeOH. 0.460 g is obtained. Yield=36.5% LCMS (A) M=458 tr=5.82 min. ¹H NMR (DMSO-d6 250 MHz): 1.32 (t, 3); 4.34 (d, 2); 4.62 (quad, 2); 6.83 (t, 1); 7.36 (m, 1); 7.6 (d, 2); 7.72 (d, 1); 7.98 (d, 1); 8.15 (d, 2); 8.4-8.69 (unresolved peak, 4); 8.98 (s, 1); 10.41 (sl, 1). IC₅₀ (HCT116)=6 nM.

### Example 6

### 2-Amino-1-ethyl-4-oxo-7-[4-(3-pyridin-3-ylmethylureido)phenyl]-1,4-dihydro[1,8]naphthyridine-3-carboxylic Acid (2-morpholin-4-ylethyl)amide (No. 30)

### 6.1: 2-Amino-7-(4-aminophenyl)-1-ethyl-4-oxo-1,4-dihydro[1,8]naphthyridine-3-carboxylic Acid Ethyl Ester

1.3 g (3.39 mmol) of compound of stage 4.1, 20 ml of acetonitrile and 0.385 g (6.78 mmol) of a 30% aqueous ammonia solution are placed in a screw-top tube. The tube is hermetically sealed and the mixture is heated at 85° C. for 48 h. After a return to ambient temperature, the resulting product is evaporated and the residue is taken up with water, the resulting product is filtered and drying is carried out under vacuum over P₂O₅. Purification is carried out by flash chromatography with a gradient of 0-10% MeOH in CH₂Cl₂. 0.5 g of product is obtained. Yield=41.8%. LCMS (A) M=352 tr=6.62 min.

### 6.2: 2-Amino-7-(4-amino-phenyl)-1-ethyl-4-oxo-1,4-dihydro-[1,8]naphthyridine-3-carboxylic Acid (2-morpholin-4-yl-ethyl)-amide

0.5 g (1.42 mmol) of compound of stage 6.1 and 3.72 ml (3.7 mmol) of 2-morpholinoethylamine are placed in a screw-top tube. The tube is hermetically sealed and the mixture is heated at 125° C. for 20 h. After a return to ambient temperature, the precipitate is taken up in CH₂Cl₂ and washed in water with a saturated NaCl solution The mixture is dried on Na₂SO₄, filtered and evaporated. Purification is carried out by flash chromatography with a gradient of 0-10% MeOH in CH₂Cl₂. 0.530 g of product is obtained. Yield=85.6%. LCMS (A) M=436 tr=5.44 min.

### 6.3: 2-Amino-1-ethyl-4-oxo-7-[4-(3-pyridin-3-ylmethylureido)phenyl]-1,4-dihydro[1,8]naphthyridine-3-carboxylic Acid (2-morpholin-4-ylethyl)amide

0.530 g (1.21 mmol) of the compound of stage 6.2 is dissolved in 50 ml of THF. 0.178 g (1.46 mmol) of DMAP is added, followed by 0.373 g (1.46 mmol) of DSC, and the mixture is stirred at ambient temperature for 18 hours. 0.41 ml (2.91 mmol) of triethylamine and 0.158 g (1.46 mmol) of (3-aminomethyl)pyridine are added. The mixture is stirred at ambient temperature for 24 h. The resulting product is evaporated and the residue is taken up with a water-CH₂Cl₂ mixture. The precipitate is filtered off and vacuum dried over P₂O₅, and the resulting product is purified by flash chromatography with a gradient of 0-10% MeOH in CH₂Cl₂. 0.080 g of product is obtained. Yield=11.5%. Mp=245° C. LCMS (A) M=570 tr=5.13 min. ¹H NMR (DMSO-d6 250 MHz): 1.27 (t, 3); 2.29-2.41 (unresolved peak, 4); 3.36 (quad, 2); 3.48-3.62 (unresolved peak, 4); 4.31 (d, 2); 4.48 (quad, 2); 6.79 (t, 1); 7.33 (dd, 1); 7.55 (d, 2); 7.69 (d, 1); 7.85 (d, 1); 8.09 (d, 2); 8.4-8.47 (unresolved peak, 2); 8.51 (s, 1); 8.9 (s, 1); 11.29 (t, 1); 11.66 (bs, 1). IC₅₀ (HCT116)=0.1 nM.

### Example 7

### 2-Amino-1-ethyl-4-oxo-7-[4-(3-pyridin-3-ylmethylureido)phenyl]-1,4-dihydroquinoline-3-carboxylic Acid Amide (No. 57)

### 7.1: 7-Bromo-1H-benzo[d][1,3]oxazine-2,4-dione

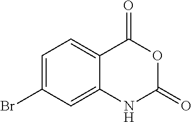

In a 250 ml round-bottomed flask, under a nitrogen atmosphere, 5.0 g (23.1 mmol) of 2-amino-4-bromobenzoic acid are dissolved in 50 ml of anhydrous dioxane. The temperature of the mixture is reduced to 0° C. by means of an ice bath. 2.3 g (7.6 mmol) of triphosgene are added dropwise. The ice bath is replaced with an oil bath and the mixture is refluxed for 16 h. After a return to ambient temperature, water (100 ml) is added and the precipitate formed is filtered off, washed with Et₂O (3×25 ml) and then dried in an oven, so as to obtain 5.6 g (23.1 mmol) of the compound in the form of a beige powder. Yield=100%. ¹H NMR DMSO d₆ (300 MHz) 7.29 (d, J=1.8 Hz, 1H); 7.41 (dd, J=8.4 Hz, J=1.8 Hz, 1H); 7.82 (d, J=8.4 Hz, 1H); 11.81 (bs, 1H).

### 7.2: 7-Bromo-1-ethylbenzo[d][1,3]oxazine-2,4-dione

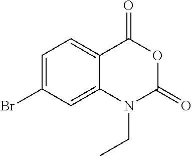

In a 250 ml round-bottomed flask, under a nitrogen atmosphere, 1.0 g (25.5 mmol) of NaH at 60% is suspended in 60 ml of anhydrous DMF. The temperature of the mixture is reduced to 0° C. by means of an ice bath. 5.6 g (23.1 mmol) of the compound of stage 7.1 are added dropwise, and then the mixture is stirred for 3 hours at ambient temperature. 2 ml (25.5 mmol) of iodoethane are added dropwise, and then the mixture is stirred for 16 h at ambient temperature. The mixture is poured into ice-cold water (100 ml) and the precipitate formed is filtered off. It is washed with water (3×25 ml) and then dried in an oven, so as to obtain 5.9 g of product (21.8 mmol) in the form of an off-white powder. Yield=95%. ¹H NMR DMSO d₆ (300 MHz) 1.20 (t, J=7.1 Hz, 3H); 4.06 (q, J=7.1 Hz, 2H); 7.50 (dd, J=8.4 Hz, J=1.5 Hz, 1H); 7.75 (d, J=1.5 Hz, 1H); 7.90 (d, J=8.4 Hz, 1H).

### 7.3: 2-Amino-7-bromo-1-ethyl-4-oxo-1,4-dihydroquinoline-3-carboxylic Acid Amide

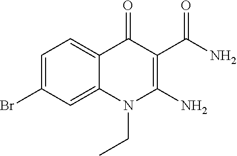

In a 100 ml round-bottomed flask, under a nitrogen atmosphere, 650 mg (16.3 mmol) of NaH at 60% are suspended in 10 ml of anhydrous DMF. The temperature of the mixture is reduced to 0° C. by means of an ice bath. A solution of 685 mg (8.1 mmol) of cyanoacetamide diluted in 10 ml of DMF is added dropwise, and then the mixture is stirred for 4 hours at ambient temperature. The temperature of the mixture is reduced to 0° C. by means of an ice bath. A solution of 2.0 g (7.4 mmol) of the compound of stage 7.2, dissolved in 10 ml of DMF, is added dropwise, and then the mixture is stirred for 16 h at ambient temperature. The reaction medium is poured into a 1 N aqueous solution of HCl at 0° C. (100 ml) and the precipitate formed is filtered off. It is dried in an oven, so as to obtain 1.8 g (5.8 mmol) of product in the form of a yellow powder. Yield=79%. ¹H NMR DMSO d₆ (300 MHz) 1.14 (t, J=7.0 Hz, 3H); 3.12 (q, J=7.0 Hz, 2H); 6.74 (d, J=8.2 Hz, 1H); 6.81 (s, 1H); 7.28 (d, J=8.2 Hz, 1H).

### 7.4: 2-Amino-7-(4-aminophenyl)-1-ethyl-4-oxo-1,4-dihydroquinoline-3-carboxylic Acid Amide

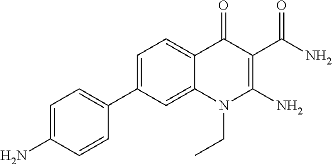

In a 50 ml round-bottomed flask, 750 mg (2.4 mmol) of the compound of stage 7.3 are dissolved in 20 ml of THF. 530 mg (2.4 mmol) of p-anilineboronic ester are added, followed by 3 ml (6.0 mmol) of a 2 M aqueous solution of Na₂CO₃. The mixture is degassed with a stream of nitrogen and then 280 mg (0.2 mmol) of Pd(PPh₃)₄ are added and the mixture is refluxed for 16 h. After a return to ambient temperature, the mixture is filtered through filter paper and the solvents are evaporated off under reduced pressure. The residue is purified by flash chromatography on a silica column (40-63 μm) (eluant: EtOAc). The pure fractions are collected and the solvent is then evaporated off under reduced pressure, so as to obtain 100 mg (0.3 mmol) of product in the form of a brown powder. Yield=13%. ¹H NMR DMSO d₆ (300 MHz) 1.31 (t, J=6.9 Hz, 3H); 4.29 (q, J=6.9 Hz, 2H); 5.39 (s, 2H); 6.69 (d, J=8.4 Hz, 2H); 7.15 (d, J=5.5 Hz, 1H); 7.45-7.60 (m, 5H); 8.18 (d, J=8.4 Hz, 1H); 10.70 (d, J=5.5 Hz, 1H).

### 7.5: Pyridin-3-ylmethylcarbamic Acid 4-nitrophenyl Ester

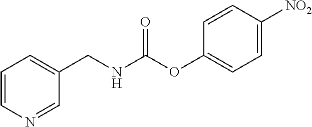

In a 100 ml round-bottomed flask, under a nitrogen atmosphere, 1.9 ml (18.5 mmol) of pyridin-3-ylmethylamine and 2.6 ml (18.5 mmol) of triethylamine are diluted in 20 ml of anhydrous diethyl ether. A solution of 3.7 g (18.5 mmol) of 4-nitrophenyl chloroformate dissolved in 30 ml of anhydrous Et₂O is added dropwise. After reaction for 30 min, a precipitate appears. The mixture is hydrolysed with 50 ml of water and then extracted with 3×20 ml of Et₂O. The organic phases are combined and then washed with 50 ml of a saturated aqueous solution of NaCl. After separation, the organic phase is dried over MgSO₄ and filtered, and the solvent is evaporated off under reduced pressure. The residue is purified by flash chromatography on a silica column (40-63 μm) (eluant: CH₂Cl₂/MeOH, 95-5). The pure fractions are collected and the solvent is then evaporated off under reduced pressure, so as to obtain 1.1 g (4.0 mmol) of the compound in the form of a white powder: Yield=22%. ¹H NMR DMSO d₆ (300 MHz): 4.32 (d, J=5.9 Hz, 2H); 7.30-7.45 (m, 3H); 7.73 (d, J=7.8 Hz, 1H); 8.24 (d, J=9.1 Hz, 2H); 8.48 (d, J=4.7 Hz, 1H); 8.54 (s, 1H); 8.62 (t, J=5.9 Hz, 1H).

### 7.6: 2-Amino-1-ethyl-4-oxo-7-[4-(pyridin-3-ylmethylureido)phenyl]-1,4-dihydroquinoline-3-carboxylic Acid Amide

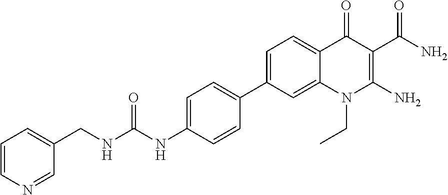

In a 25 ml round-bottomed flask, under a nitrogen atmosphere, 85 mg (0.3 mmol) of the compound of stage 7.5 are dissolved in 10 ml of absolute ethanol. 100 mg (0.3 mmol) of the compound of stage 7.4 are added and the mixture is refluxed for 16 hours. The solvent is evaporated off under reduced pressure. The residue is purified by flash chromatography on a silica column (40-63 μm) (eluant: EtOAc/MeOH, 85-15). The pure fractions are collected and the solvent is then evaporated off under reduced pressure, so as to obtain 25 mg (0.05 mmol) of the product in the form of a pale orange powder. Yield=18%; Mp=242° C. ¹H NMR DMSO-d₆ (300 MHz) 1.32 (t, J=6.6 Hz, 3H); 4.34 (m, 4H); 6.85 (t, J=5.2 Hz, 1H); 7.19 (d, J=5.2 Hz, 1H); 7.33-7.40 (dd, J=7.7 Hz, J=4.8 Hz, 1H); 7.54-7.58 (m, 3H); 7.68-7.74 (m, 4H); 8.23 (d, J=8.3 Hz, 1H); 8.46 (dd, J=4.8 Hz, J=1.4 Hz, 1H); 8.54 (d, J=1.8 Hz, 1H); 8.91 (s, 1H); 10.68 (d, J=5.0 Hz, 1H). IC₆₀ (HCT116)=0.26 nM.

### Example 8

### 1-Ethyl-4-oxo-7-[4-(3-pyridin-3-ylmethylureido)phenyl]-2-(2-pyrrolidin-1-ylethylamino)-1,4-dihydroquinoline-3-carboxylic Acid (2-pyrrolidin-1-ylethyl)amide Hydrochloride (No. 61)

### 8.1: 4-Bromo-2-fluorobenzoyl Chloride

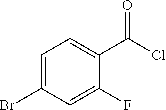

In a 500 ml round-bottomed flask, under a nitrogen atmosphere, 20.0 g (91.3 mmol) of 2-fluoro-4-bromobenzoic acid are dissolved in 250 ml of anhydrous dichloromethane. 10.7 ml (123.3 mmol) of oxalyl chloride are added dropwise, followed by the addition of DMF, dropwise, until evolution of gas. The solution is stirred for 4 h at ambient temperature and then the solvents are evaporated off under reduced pressure, so as to obtain 21.7 g (91.3 mmol) of crude product in the form of a yellow oil which is used directly for the next stage.

### 8.2: 3-(4-Bromo-2-fluorophenyl)-3-oxopropionic Acid Ethyl Ester

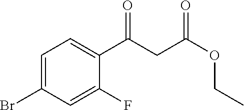

In a 500 ml round-bottomed flask, under a nitrogen atmosphere, 26.5 g (200.9 mmol) of monoethyl malonate are dissolved in 100 ml of anhydrous THF. The temperature of the mixture is reduced to 0° C. by means of an ice bath, and then 161 ml of a 2.5N solution of n-BuLi in THF are added dropwise. At the end of the addition, the temperature of the mixture is decreased to −50° C. by means of a bath of dry ice in acetone. A solution of 21.7 g (91.3 mmol) of the compound 8.1, dissolved in 100 ml of anhydrous THF, is added dropwise. The mixture is stirred for 16 h at ambient temperature. The mixture is hydrolysed with a 1N aqueous solution of HCl (250 ml) and extracted with ethyl acetate (4×100 ml). The organic phases are combined and then washed with 150 ml of a saturated aqueous solution of NaCl. After separation, the organic phase is dried over MgSO₄ and filtered, and the solvent is evaporated off under reduced pressure. The residue is purified by flash chromatography on a silica column (40-63 μm) (eluant: cyclohexane/EtOAc, 95-5). The pure fractions are collected and then the solvent is evaporated off under reduced pressure, so as to obtain 22.7 g (78.5 mmol) of product in the form of a colourless oil. This product is a mixture of the ketone and enol forms (6/4) of the product: Yield=86%. ¹H NMR CDCl₃ (300 MHz): 1.24 (t, J=7.1 Hz, 3H enol); 1.38 (t, J=7.1 Hz, 3H ketone); 3.94 (d, J=3.6 Hz, 2H ketone); 4.20 (q, J=7.1 Hz, 2H enol); 4.37 (q, J=7.1 Hz, 2H ketone); 5.81 (s, 1H enol); 7.25-7.45 (m, 2H enol, 2H ketone); 7.70-7.85 (m, 1H enol, 1H ketone); 12.67 (s, 1H enol).

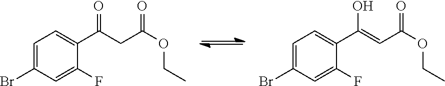

### 8.3: 2-(4-Bromo-2-fluorobenzoyl)-3,3-bismethylsulfanylacrylic Acid Ethyl Ester

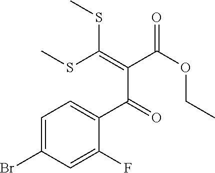

In a 250 ml round-bottomed flask, under a nitrogen atmosphere, 17.7 g (61.2 mmol) of compound of stage 8.2 are dissolved, dropwise, in 125 ml of anhydrous DMF. 25.4 g (183.6 mmol) of K₂CO₃ are added and the mixture is stirred for 15 min at ambient temperature. The temperature of the mixture is reduced to 0° C. by means of an ice bath. A solution of 4.5 ml (73.4 mmol) of CS₂ diluted in 10 ml of DMF is added dropwise, and then the mixture is stirred for 15 min at 0° C. before rapidly adding 11.5 ml (183.6 mmol) of methyl iodide. The mixture is stirred while allowing it to return to ambient temperature, and stirring is continued for 2 h. The solvent is evaporated off under reduced pressure, and the residue is purified by flash chromatography on silica gel (40-63 μm) (eluant: cyclohexane/EtOAc, 90-10). The pure fractions are collected and the solvent is then evaporated off under reduced pressure, so as to obtain 7.5 g (19.6 mmol) of a product in the form of a beige powder. Yield=32%. ¹H NMR CDCl₃ (300 MHz) 1.12 (t, J=7.1 Hz, 3H); 2.37 (bs, 6H); 4.15 (q, J=7.1 Hz, 2H); 7.30 (dd, J=8.4 Hz, J=1.6 Hz, 1H); 7.39 (dd, J=8.4 Hz, J=1.6 Hz, 1H); 7.79 (t, J=8.2 Hz, 1H).

### 8.4: 2-(4-Bromo-2-fluorobenzoyl)-3-ethylamino-3-methylsulfanylacrylic Acid Ethyl Ester

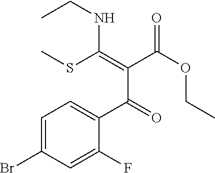

In a 250 ml round-bottomed flask, 7.5 g (19.1 mmol) of compound of stage 8.3 are dissolved in 100 ml of THF. The temperature of the mixture is reduced to 0° C. by means of an ice bath. 10 ml (20.0 mmol) of 2 Methylamine in THF are added in a single step. The mixture is stirred at ambient temperature for 24 h. The solvent is evaporated off under reduced pressure and the residue is purified by flash chromatography on silica gel (40-63 μm) (eluant: cyclohexane/EtOAc, 1-1). The pure fractions are collected and the solvent is then evaporated off under reduced pressure, so as to obtain 3.1 g (10.5 mmol) of compound in the form of a white powder. Yield=55%. ¹H NMR CDCl₃ (300 MHz) 0.90 (t, J=7.1 Hz, 3H); 1.34 (t, J=7.2 Hz, 3H); 2.44 (s, 3H); 3.63 (q, J=7.1 Hz, 2H); 3.91 (q, J=7.2 Hz, 2H); 7.15-7.30 (m, 3H); 11.71 (bs, 1H).

### 8.5: 7-Bromo-1-ethyl-2-methylsulfanyl-4-oxo-1,4-dihydroquinoline-3-carboxylic Acid Ethyl Ester

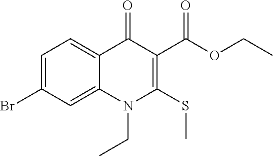

In a 100 ml round-bottomed flask, under a nitrogen atmosphere, 3.1 g (7.8 mmol) of compound of stage 8.4 are dissolved in 20 ml of anhydrous DMF. 1.3 g (9.4 mmol) of K₂CO₃ are added in one portion, and then the mixture is stirred for 16 h at 70° C. The mixture is diluted with 100 ml of water and then extracted with 3×50 ml of EtOAc. The organic phases are combined and then washed with 50 ml of a saturated aqueous solution of NaCl. After separation, the organic phase is dried over MgSO₄ and filtered, and the solvent is evaporated off under reduced pressure. The residue is purified by flash chromatography on a silica column (40-63 μm) (eluant: cyclohexane/EtOAc, 1-1). The pure fractions are collected and then the solvent is evaporated off under reduced pressure, so as to obtain 2.7 g (7.2 mmol) of compound in the form of a white powder. Yield=92%. ¹H NMR CDCl₃ (300 MHz) 1.40 (t, J=7.1 Hz, 3H); 1.47 (t, J=7.1 Hz, 3H); 2.57 (s, 3H); 4.43 (q, J=7.1 Hz, 2H); 4.65 (q, J=7.1 Hz, 2H); 7.48 (dd, J=8.6 Hz, J=1.6 Hz, 1H); 7.72 (d, J=1.6 Hz, 1H); 8.28 (d, J=8.6 Hz, 1H).

### 8.6: 7-(4-Aminophenyl)-1-ethyl-2-methylsulfanyl-4-oxo-1,4-dihydroquinoline-3-carboxylic Acid Ethyl Ester

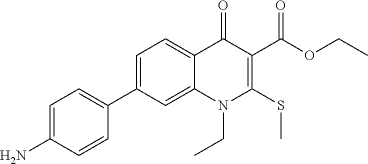

In a 250 ml round-bottomed flask, 2.7 g (7.2 mmol) of compound of stage 8.5 are dissolved in 100 ml of THF. 530 mg (7.9 mmol) of p-anilineboronic ester are added, followed by 9 ml (18.0 mmol) of a 2M aqueous solution of Na₂CO₃. The mixture is degassed with a stream of nitrogen, and then 840 mg (0.7 mmol) of Pd(PPh₃)₄ are added and the mixture is refluxed for 16 h. After a return to ambient temperature, the mixture is filtered through filter paper and the solvents are evaporated off under reduced pressure. The residue is taken up in 100 ml of water and then extracted with 3×50 ml of EtOAc. The organic phases are combined and then washed with 50 ml of a saturated aqueous solution of NaCl. After separation, the organic phase is dried over MgSO₄ and filtered, and the solvent is evaporated off under reduced pressure. The residue is purified by flash chromatography on a silica column (40-63 μm) (eluant: EtOAc). The pure fractions are collected and then the solvent is evaporated off under reduced pressure, so as to obtain 1.1 g (2.9 mmol) of compound in the form of a yellow powder. Yield=40%. ¹H NMR CDCl₃ (300 MHz) 1.40 (t, J=6.8 Hz, 3H); 1.48 (t, J=6.8 Hz, 3H); 2.57 (s, 3H); 4.43 (q, J=6.8 Hz, 2H); 4.74 (q, J=6.8 Hz, 2H); 6.87 (d, J=8.5 Hz, 2H); 7.46 (d, J=8.5 Hz, 2H); 7.53 (d, J=8.4 Hz, 1H); 7.61 (s, 1H); 8.42 (d, J=8.4 Hz, 1H).

### 8.7: 7-(4-Aminophenyl)-1-ethyl-4-oxo-2-(2-pyrrolidin-1-ylethylamino)-1,4-dihydroquinoline-3-carboxylic Acid (2-pyrrolidin-1-ylethyl)amide

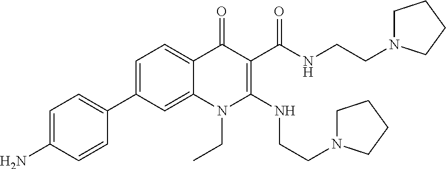

In a sealed 5 ml tube, 270 mg (0.7 mmol) of compound of stage 8.6 are mixed into 2 ml of 1-(2-aminoethyl)pyrrolidine. The mixture is stirred for 16 h at 135° C. After a return to ambient temperature, the excess amine is evaporated off under reduced pressure and the residue is then purified by flash chromatography on a silica column (40-63 μm) (eluant: EtOAc/MeOH/TEA: 87-10-3). The pure fractions are collected and the solvent is then evaporated off under reduced pressure, so as to obtain 260 mg (0.5 mmol) of compound in the form of a colourless oil. Yield=72%. ¹H NMR CDCl₃ (300 MHz) 1.24 (t, J=6.8 Hz, 3H); 1.76 (m, 4H); 1.84 (m, 4H); 2.58 (m, 4H); 2.72 (m, 4H); 2.81 (m, 4H); 3.48 (m, 2H); 3.62 (m, 2H); 4.33 (q, J=6.8 Hz, 2H); 6.78 (d, J=8.4 Hz, 2H); 7.40-7.50 (m, 4H); 8.33 (d, J=8.4 Hz, 1H); 11.14 (s, 1H); 11.35 (s, 1H).

### 8.8: 7-[4-(Pyridin-3-ylmethylureido)phenyl]-1-ethyl-4-oxo-2-(2-pyrrolidin-1-ylethylamino)-1,4-dihydroquinoline-3-carboxylic Acid (2-pyrrolidin-1-ylethyl)amide Hydrochloride Salt

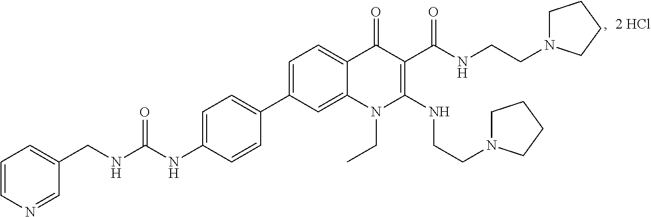

In a 25 ml round-bottomed flask, under a nitrogen atmosphere, 260 mg (0.5 mmol) of compound of stage 8.7 are dissolved in 10 ml of anhydrous THF. 193 mg (0.7 mmol) of N,N′-disuccinimidyl carbonate and 93 mg (0.7 mmol) of dimethylaminopyridine are added and the mixture is then stirred for 16 hours at ambient temperature. 240 μl (1.5 mmol) of triethylamine and a solution of 82 mg (0.7 mmol) of pyridin-3-ylmethylamine dissolved in 2 ml of anhydrous THF are added and then the mixture is stirred for 6 h at ambient temperature. The solvent is evaporated off under reduced pressure. The residue is purified by flash chromatography on a neutral alumina column (eluant: EtOAc/MeOH, 90-10). The pure fractions are collected and the solvent is then evaporated off under reduced pressure. The product is dissolved in 2 ml of EtOAc and then a 2N solution of HCl in EtOAc is added, dropwise, until complete formation of the salt. The solvent is evaporated off under reduced pressure, and then the solid is taken up in EtOAc, triturated and filtered, so as to obtain 100 mg (0.1 mmol) of compound in the form of a white solid. Yield=30%; Mp=130-135° C. ¹H NMR DMSO-d₆ (300 MHz) 1.10-1.25 (m, 3H); 1.75-2.10 (m, 8H); 2.90-3.10 (m, 4H); 3.25-3.85 (m, 12H); 4.40-4.55 (m, 4H); 7.43 (m, 1H); 7.50-7.78 (m, 5H); 7.85 (s, 1H); 8.04 (m, 1H); 8.18 (m, 1H); 8.50 (m, 1H); 8.84 (m, 2H); 9.63 (s, 1H); 10.50-10.80 (m, 2H); 11.19 (s, 1H); 11.36 (bs, 1H). ¹³C NMR DMSO-d₆ (75 MHz) 14.57, 21.26, 23.18, 35.23, 44.20, 45.23, 53.17, 53.56, 53.79, 60.23, 99.19, 115.44, 118.43, 122.45, 124.23, 126.89, 127.32, 128.11, 131.91, 139.63, 140.97, 141.08, 141.40, 144.33, 144.69, 155.93, 162.93, 169.67, 170.79, 175.54. IC₅₀ (HCT116)=0.1 nM.

### Example 9

### 7-Amino-8-ethyl-5-oxo-2-[4-(3-pyridin-3-ylmethylureido)phenyl]-5,8-dihydropyrido[2,3-d]pyrimidine-6-carboxylic Acid Methylamide (No. 53)

### 9.1: 2,4-Dihydroxypyrimidine-5-carbonyl Chloride

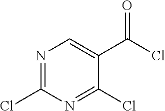

In a 250 ml round-bottomed flask, under a nitrogen atmosphere, 10.0 g (64.0 mmol) of 2,4-dihydroxypyrimidine-5-carboxylic acid are dissolved in 46 ml (500.0 mmol) of POCl₃. The temperature of the mixture is reduced to 0° C. by means of an ice bath, and then 47.7 g (230 mmol) of PCl₅ are added in small portions. The solution is stirred for 16 h at reflux and the solvents are then evaporated off under reduced pressure. The residue is taken up and triturated in 100 ml of toluene and then filtered. This operation is repeated three times and then the filtrate is evaporated under reduced pressure, so as to give 13.5 g (64.0 mmol) of compound in the form of a yellow oil which is used directly for the next stage. Yield=100%.

### 9.2: 2,4-Dichloropyrimidine-5-carboxylic Acid Ethyl Ester

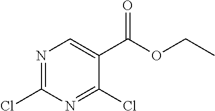

In a 250 ml round-bottomed flask, under a nitrogen atmosphere, 13.5 g (64.0 mmol) of above compound are dissolved in 100 ml of anhydrous THF. 15 ml of absolute ethanol are added and the mixture is stirred for 10 min at ambient temperature. The mixture is diluted with a saturated aqueous solution of K₂CO₃ (100 ml) and extracted with ethyl acetate (4×100 ml). The organic phases are combined and then washed with 150 ml of a saturated aqueous solution of NaCl. After separation, the organic phase is dried over MgSO₄ and filtered, and the solvent is evaporated off under reduced pressure, so as to obtain 14.0 g (63.3 mmol) of compound in the form of an orange oil. Yield=99%. ¹H NMR DMSO d₆ (300 MHz): 1.34 (t, J=7.1 Hz, 3H); 4.37 (q, J=7.1 Hz, 2H); 9.16 (s, 1H).

### 9.3. 2-Chloro-4-ethylaminopyrimidine-5-carboxylic Acid Ethyl Ester

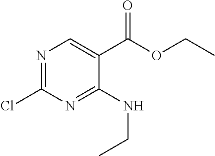

In a 250 ml round-bottomed flask, under a nitrogen atmosphere, 14.0 g (63.3 mmol) of compound of the preceding stage are dissolved in 150 ml of anhydrous THF. 13 ml (95.0 mmol) of triethylamine are added, followed by 32 ml (64.0 mmol) of 2M ethylamine in THF, and the mixture is stirred for 16 h at ambient temperature. The precipitate formed is filtered off and the filtrate is evaporated under reduced pressure. The residue is purified by flash chromatography on a silica column (40-63 μm) (eluant: cyclohexane/EtOAc, 1-1). The pure fractions are collected and the solvent is then evaporated off under reduced pressure, so as to obtain 9.2 g (39.9 mmol) of compound in the form of a white powder. Yield=63%. ¹H NMR DMSO d₆ (300 MHz): 1.15 (t, J=7.2 Hz, 3H); 1.31 (t, J=7.1 Hz, 3H); 3.47 (m, 2H); 4.37 (q, J=7.1 Hz, 2H); 8.50 (bs, 1H); 8.59 (s, 1H).

### 9.4: 2-Chloro-4-ethylaminopyrimidine-5-carboxylic Acid

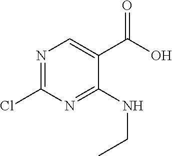

In a 500 ml round-bottomed flask, 9.2 g (39.9 mmol) of compound of stage 9.3 are dissolved in 250 ml of THF. 100 ml of water followed by 2.5 g (60.0 mmol) of lithium hydroxide monohydrate are added. The mixture is stirred at ambient temperature for 24 h. The THF is evaporated off under reduced pressure, and the solution is acidified with a 1N aqueous solution of HCl until complete formation of the precipitate. The precipitate formed is filtered off and is then dried in an oven, so as to obtain 8.0 g (39.5 mmol) of the compound in the form of a white powder. Yield=99%. ¹H NMR DMSO d₆ (300 MHz): 1.15 (t, J=7.2 Hz, 3H); 3.45 (m, 2H); 8.55 (s, 1H); 8.65 (bs, 1H).

### 9.5. 2-Chloro-4-ethylaminopyrimidine-5-carbonyl Fluoride

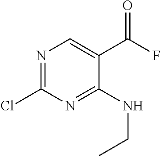

In a 250 ml round-bottomed flask, under a nitrogen atmosphere, 4.0 g (20.0 mmol) of compound of stage 9.4 are dissolved in 120 ml of anhydrous DCM. 2.8 ml (20.0 mmol) of triethylamine are added, followed by 2.5 ml (30.0 mmol) of cyanuric fluoride, and the mixture is stirred for 16 h at ambient temperature. The mixture is hydrolysed with 50 ml of an ice-cold saturated solution of NaHCO₃ and then extracted with 3×25 ml of EtOAc. The organic phases are combined and then washed with 25 ml of a saturated aqueous solution of NaCl. After separation, the organic phase is dried over MgSO₄ and filtered, and the solvent is evaporated off under reduced pressure, so as to obtain 3.2 g (15.8 mmol) of compound in the form of a yellow oil which is used directly for the next stage. Yield=79%.

### 9.6: 7-Amino-2-chloro-8-ethyl-5-oxo-5,8-dihydropyrido[2,3-d]pyrimidine-6-carboxylic Acid Methylamide

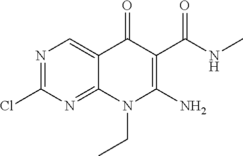

In a 250 ml round-bottomed flask, under a nitrogen atmosphere, 1.3 g (33.0 mmol) of NaH at 60% are suspended in 35 ml of anhydrous DMF. The temperature of the mixture is reduced to 0° C. by means of an ice bath. 1.6 g (16.5 mmol) of 2-cyano-N-methylacetamide are added and the mixture is then stirred for 15 min at 0° C. and 1 h at ambient temperature. The temperature of the mixture is reduced to 0° C. by means of an ice bath, before said mixture is added dropwise to a solution, at 0° C., of 3.2 g (15.8 mmol) of the compound of stage 9.5 dissolved in 35 ml of DMF. The mixture is stirred for 2 hours at ambient temperature and then 660 mg (16.5 mmol) of NaH at 60% are added. The stirring of the mixture is continued for 2 h at ambient temperature and then the mixture is poured into ice-cold water (50 ml) and the DMF is evaporated off under reduced pressure. The precipitate formed is filtered off and washed with water (2×25 ml) and the resulting product is dried in an oven, so as to obtain 2.3 g (8.4 mmol) of compound in the form of a white powder. Yield=53%. ¹H NMR DMSO d₆ (300 MHz): 1.23 (t, J=7.1 Hz, 3H); 2.80 (d, J=4.7 Hz, 3H); 4.34 (q, J=7.1 Hz, 2H); 8.42 (bs, 1H); 9.11 (s, 1H); 10.71 (d, J=4.7 Hz, 1H); 11.96 (bs, 1H).

### 9.7: 7-Amino-2-[4-(pyridin-3-ylmethylureido)phenyl]-8-ethyl-5-oxo-5,8-dihydropyrido[2,3-d]pyrimidine-6-carboxylic Acid Methylamide

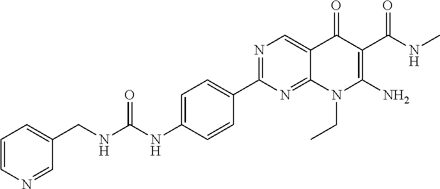

In a 50 ml round-bottomed flask, 300 mg (1.1 mmol) of compound of stage 9.6 are dissolved in 15 ml of THF. 564 mg (1.6 mmol) of 1-pyridin-3-ylmethyl-3-[4-(4,4,5,5-tetramethyl[1,3,2]dioxaborolan-2-yl)phenyl]urea are added, followed by 1.1 ml (2.2 mmol) with 2M aqueous solution of Na₂CO₃. The mixture is degassed with a stream of nitrogen and then 612 mg (0.5 mmol) of Pd(PPh₃)₄ are added and the mixture is refluxed for 4 h. After a return to ambient temperature, the mixture is filtered and the precipitate is washed with ethanol. After recrystallization from ethanol and washing with ethyl ether, 462 mg (1.0 mmol) of compound are isolated in the form of a yellow powder. Yield=92%; Mp=250° C. ¹H NMR DMSO-d₆ (300 MHz) 1.26 (t, J=6.8 Hz, 3H); 2.76 (d, J=4.6 Hz, 3H); 4.33 (d, J=5.4 Hz, 2H); 4.50 (d, J=7.1 Hz, 2H); 7.20-7.30 (m, 1H); 7.33-7.38 (m, 1H); 7.59 (d, J=8.7 Hz, 2H); 7.72 (d, J=7.8 Hz, 1H); 8.33 (d, J=8.7 Hz, 2H); 8.44 (dd, J=4.8 Hz, J=1.5 Hz, 1H); 8.53 (5, 1H); 9.11 (s, 1H); 9.37 (bs, 1H). LCMS (D) tr=5.93 min. IC₅₀ (HCT116)=6 nM.

### Example 10

### Compound No. 27

¹H NMR DMSO-d₆ (300 MHz) 1.31 (t, J=6.7 Hz, 3H); 2.80 (d, J=4.5 Hz, 3H); 4.20-4.40 (m, 4H); 6.81 (t, J=5.7 Hz, 1H); 7.37 (m, 1H); 7.56 (m, 3H); 7.65-7.75 (m, 4H); 8.23 (d, J=8.1 Hz, 1H); 8.46 (d, J=4.8 Hz, 1H); 8.54 (s, 1H); 8.90 (s, 1H); 11.31 (m, 1H). LCMS (D): tr=6.28 min. IC₅₀ (HCT116)=0.1 nM.

### Example 11

### Compound No. 56

¹H NMR DMSO-d₆ (300 MHz) 1.32 (t, J=6.6 Hz, 3H); 4.34 (m, 4H); 6.85 (t, J=5.2 Hz, 1H); 7.19 (d, J=5.2 Hz, 1H); 7.33-7.40 (dd, J=7.7 Hz, J=4.8 Hz, 1H); 7.54-7.58 (m, 3H); 7.68-7.74 (m, 4H); 8.23 (d, J=8.3 Hz, 1H); 8.46 (dd, J=4.8 Hz, J=1.4 Hz, 1H); 8.54 (d, J=1.8 Hz, 1H); 8.91 (s, 1H); 10.68 (d, J=5.0 Hz, 1H). LCMS (D): tr=5.71 min. IC₅₀ (HCT116)=0.19 nM.

### Example 12

### Compound No. 59

¹H NMR CDCl₃ (300 MHz) 1.18 (t, J=6.9 Hz, 3H); 1.50-1.70 (m, 2H); 1.80-2.10 (m, 6H); 3.35-3.50 (m, 3H); 3.58-3.69 (m, 1H); 3.70-3.82 (m, 2H); 3.82-3.96 (m, 2H); 4.03-4.16 (m, 2H); 4.28 (q, J=7.1 Hz, 2H); 4.46 (d, J=5.7 Hz, 2H); 6.32 (t, J=5.8 Hz, 1H); 7.20-7.30 (m, 1H); 7.32-7.50 (m, 6H); 7.75 (d, J=7.8 Hz, 1H); 8.00 (5, 1H); 8.27 (d, J=8.3 Hz, 1H); 8.46 (dd, J=4.8 Hz, J=1.4 Hz, 1H); 8.58 (m, 1H); 11.30-11.45 (m, 2H). ¹³H NMR CHCl₃-d (75 MHz) 8.62, 138.2, 25.83, 29.06, 29.26, 41.4, 43.00, 45.68, 46.04, 52.92, 69.23, 68.53, 77.96, 99.07, 114.79, 119.36, 122.41, 123.87, 124.88, 127.23, 127.76, 133.72, 135.92, 136.39, 139.39, 139.95, 144.41, 147.57, 148.09, 155.90, 164.46, 169.37, 176.26. LCMS (D): tr=6.52 min. IC₅₀ (HCT116)=0.47 nM.

### Example 13

### Compound No. 60

¹H NMR CHCl₃-d (300 MHz) 0.90-1.00 (m, 12H); 1.00-1.10 (m, 12H); 1.17 (t, J=6.9 Hz, 3H); 2.62-2.77 (m, 4H); 2.90-3.14 (m, 4H); 3.25-3.35 (m, 2H); 3.35-3.48 (m, 2H); 4.22-4.33 (m, 2H); 4.44 (d, J=5.4 Hz, 2H); 6.20 (m, 1H); 7.20 (m, 1H); 7.35-7.50 (m, 6H); 7.67 (d, J=7.8 Hz, 1H); 8.02 (bs, 1H); 8.27 (d, J=8.6 Hz, 1H); 8.46 (d, J=4.3 Hz, 1H); 8.54 (s, 1H); 10.81 (bs, 1H); 11.05 (bs, 1H). LCMS (D): tr=5.12 min. IC₅₀ (HCT116)=0.1 nM.

TABLE I
(I)

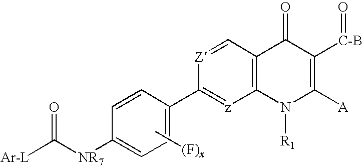

| | | | | | | | | R₆ | | LC² | | |
|---|---|---|---|---|---|---|---|---|---|---|---|---|
| | | | | | | R₂/R₃ | R₄R₅ | (when B = | MS | tr in min | Mp³ or | |
| No. | Ar | L¹ | R₇ | Z/Z′ | R₁ | (A = —NR₂R₃) | (when B = —NR₄R₅) | —OR₆) | (MH+) | (method) | NMR | comment |
| 1 | Ar₁ | CH₂NH | H | N/ CH | Et | H/H | H/Me | | 472 | 5.74 (A) | NMR | cf. ex. 1 hydrochloride |
| 2 | Ar₁ | NH | H | N/ CH | Et | H/H | H/Me | | 458 | | >260° C. | hydrochloride |
| 3 | Ar₁ | CH₂CH₂NH | H | N/ CH | Et | H/H | H/Me | | 486 | | 198- 200° C. | hydrochloride |
| 4 | Ar₁ (x = 1)⁴ | CH₂NH | H | N/ CH | Et | H/H | H/Me | | 490 | | 194 | trifluoro- methane- sulfonate |
| 5 | Ar₃ | NH | H | N/ CH | Et | H/H | H/Me | | 473 | | >260° C. | hydrochloride |
| 6 | Ar₁ | CH₂NH | H | N/ CH | Et | H/H | H/Me | | 458 | 5.55 (A) | | hydrochloride |
| 7 | Ar₁ | CH═CH | H | N/ CH | Et | H/H | H/Me | | 469 | 7.05 (A) | NMR | cf. ex.2 |
| 8 | Ar₁ | CH₂NH | H | N/ CH | CH₂CH₂N Me₂ | H/H | H/Me | | 515 | 4.84 (A) | | |
| 9 | Ar₁ | CH₂NH | H | N/ CH | Et | H/H | H/H | | 458 | 5.50 (A) | | |
| 10 | Ar₁ | CH₂NH | H | N/ CH | Et | H/H | | Et | 487 | 5.68 (A) | NMR | cf. ex.3 |
| 11 | Ar₁ | CH₂NH | H | N/ CH | Et | H/H | H/Me | | 486 | 6.49 (A) | | |
| 12 | Ar₁ | CH₂Me | H | N/ CH | Et | H/H | H/Me | | 486 | 6.11 (A) | | |
| 13 | Ar₁ | CH₂NH | H | N/ CH | Et | H/H | | H | 459 | 5.82 (A) | NMR | cf. ex.5 |
| 14 | Ar₁ | CH₂NH | H | N/ CH | Et | H/H | Me/Me | | 486 | 5.06 (A) | | |
| 15 | Ar₁ | CH₂NH | H | N/ CH | Et | H/H | H/—CH₂CH₂NMe₂ | | 529 | 4.93 (A) | | |
| 16 | Ar₁ | CH₂CH(NHC OOt-Bu) | H | N/ CH | Et | H/H | H/Me | | 586 | 6.86 (A) | | |
| 17 | Ar₄ | CH₂NH | H | N/ CH | Et | H/H | H/Me | | 478 | 7.00 (A) | | |
| 18 | Ar₁ | CH₂O | H | N/ CH | Et | H/H | H/Me | | 473 | 6.93 (A) | | |
| 19 | Ar₁ | CH₂NH | Me | N/ CH | Et | H/H | H/Me | | 486 | 6.08 (A) | | |
| 20 | Ar₁ | CH₂NH | H | N/ CH | Et | H/H | H/ —(CH₂)₆NHCOOt- Bu | | 657 | 7.53 (A) | | |
| 21 | Ar₃ | CH₂NH | H | N/ CH | Et | H/H | H/Me | | 487 | 5.74 (A) | | |
| 22 | Ar₁ | CH₂NH | H | N/ CH | Et | H/ —CH₂CH₂NMe₂ | H/—CH₂CH₂NMe₂ | | 600 | 4.56 (A) | | |
| 23 | Ar₁ | CH₂NMe | H | N/ CH | Et | H/H | H/H | | 472 | 5.85 (A) | | |
| 24 | Ar₁ | CH₂NH | H | N/ CH | (CH₂)₆NH COOt-Bu | H/H | H/Me | | 643 | 7.08 (A) | | |
| 25 | Ar₁ | CH₂NH | H | N/ CH | —(CH₂)₆NH₂ | H/H | H/Me | | 543 | 5.43 (A) | | trifluoro- methane- sulfonate |
| 26 | Ar₁ | CH₂NH | H | N/ CH | Et | H/H | H/—(CH₂)₆NH₂ | | 557 | 5.44 (A) | | |
| 27 | Ar₁ | CH₂NH | H | N/ CH | Et | H/H | H/Me | | 471 | 6.28 (D) | NMR | |
| 28 | Ar₁ | CH₂NH | H | N/ CH | Et | H/H | H/Me | | 486 | 6.13 (A) | | |
| 29 | Ar₁ | CH₂NH | H | N/ CH | Et | H/Me | 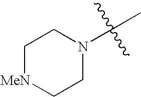 | | 541 | 4.51 (A) | | |
| 30 | Ar₁ | CH₂NH | H | N/ CH | Et | H/Me | 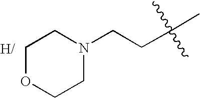 | | 571 | 5.13 (A) | 245° C./ NMR | cf. ex.6 |
| 31 | Ar₁ | CH₂NH | H | N/ CH | Et | 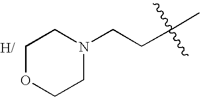 | 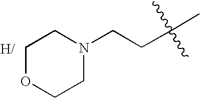 | | 684 | 6.01 (B) | | |
| 32 | Ar₁ | CH₂NH | H | N/ CH | Et | H/H | H/ —[(CH₂)₂O]₇CH₂NH₂ | | 809 | 5.59 (A) | | trifluoro- methane- sulfonate |
| 33 | Ar₁ | CH₂NH | H | N/ CH | 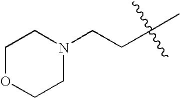 | H/H | H/Me | | 557 | 5.12 (A) | | |
| 34 | Ar₁ | CH₂NH | H | N/ CH | Et | 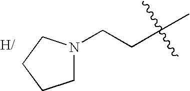 | 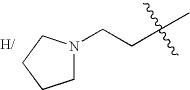 | | 652 | 4.69 (A) | | |
| 35 | Ar₁ | CH₂NH | H | N/ CH | CH₂CH₂N Me₂ | H/H | H/—CH₂CH₂NMe₂ | | 572 | 4.43 (A) | | |
| 36 | Ar₁ | CH₂NH | H | N/ CH | Et | H/H | 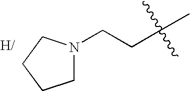 | | 555 | 5.35 (A) | | |
| 37 | Ar₁ | CH₂NH | H | N/ CH | 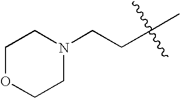 | H/H | H/H | | 543 | 5.02 (A) | | |
| 38 | Ar₁ | CH₂Me | H | N/ CH | 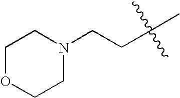 | H/H | H/Me | | 571 | 5.50 (A) | | |
| 39 | Ar₁ | CH₂Me | H | N/ CH | Et | H/H | 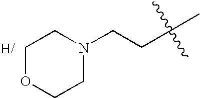 | | 585 | 5.24 (A) | | |
| 40 | Ar₁ | CH₂NH | H | N/ CH | Et | H/H | H/—[(CH₂)₃NH]₃—H | | 629 | 4.51 (A) | | trifluoro- methane- sulfonate |
| 41 | Ar₁ | CH₂NH | H | N/ CH | 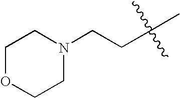 | H/H | H/—CH₂CH₂NH₂ | | 586 | 4.65 (A) | | trifluoro- methane- sulfonate |
| 42 | Ar₁ | CH₂NH | H | N/ CH | Et | 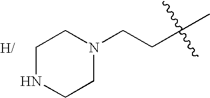 | 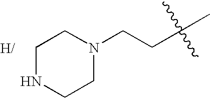 | | 682 | 4.62 (A) | | trifluoro- methane- sulfonate |
| 43 | Ar₁ | CH₂NH | H | N/ CH | Et | H/H | 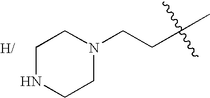 | | 570 | 4.97 (A) | | trifluoro- methane- sulfonate |
| 44 | Ar₁ | CH₂NH | H | N/ CH | Et | 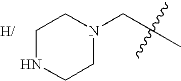 | 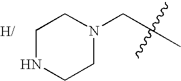 | | 652 | 5.03 (A) | | trifluoro- methane- sulfonate |
| 45 | Ar₁ | CH₂Me | H | N/ CH | Et | 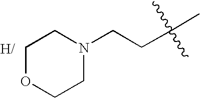 | 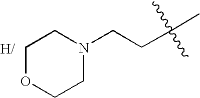 | | 698 | 4.80 (A) | | |
| 46 | Ar₁ | CH₂NH | H | N/ CH | Et | 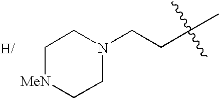 | 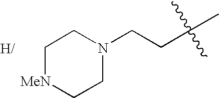 | | 710 | 4.86 ((A) | NMR | cf. ex.4 |
| 47 | Ar₁ | CH₂NH | H | N/ CH | 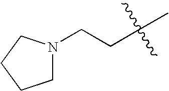 | H/H | H/Me | | 541 | 5.15 (A) | | |
| 48 | Ar₁ | CH₂Me | H | N/ CH | Et | H/H | 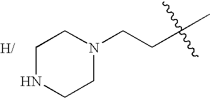 | | 584 | 5.24 (A) | | trifluoro- methane- sulfonate |
| 49 | Ar₁ | CH₂NH | H | N/ CH | Et | H/H | 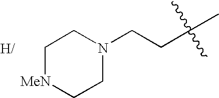 | | 584 | 5.20 (A) | | |
| 50 | Ar₁ | CH₂NH | H | N/ CH | Et | H/—CH₂CH₂NH₂ | H/—CH₂CH₂NH₂ | | 544 | 4.60 (A) | | trifluoro- methane- sulfonate |
| 51 | Ar₁ | CH₂NH | H | N/ CH | Et | H/H | 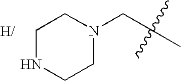 | | 555 | 5.33 (A) | | trifluoro- methane- sulfonate |
| 52 | Ar₁ | CH₂NH | H | N/ CH | 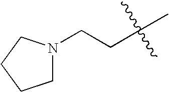 | H/H | H/H | | 527 | 6.34 (C) | | |
| 53 | Ar₁ | CH₂NH | H | N/N | Et | H/H | H/Me | | 473 | | 250° C./ NMR | cf.ex.9 |
| 54 | Ar₁ | CH₂NH | H | N/ CH | Et | H/H | 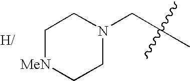 | | 569 | 4.97 (A) | | |
| 55 | Ar₁ | CH₂NH | H | N/ CH | Et | H/—CH₂CH₂N(i-Pr)₂ | H/—CH₂CH₂N(i-Pr)₂ | | 712 | 5.08 (A) | | |
| 56 | Ar₁ | CH₂NH | H | CH/ CH | Et | H/H | H/H | | 457 | 5.71 (D) | | |
| 57 | Ar₁ | CH₂NH | H | N/ CH | Et | 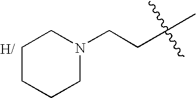 | 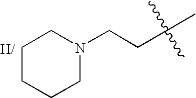 | | 680 | 4.90 (D) | 242° C./ NMR | cf.ex.7 |
| 58 | Ar₁ | CH₂NH | H | N/ CH | Et | 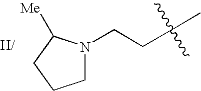 | 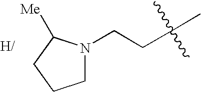 | | 680 | 4.71 (A) | | |
| 59 | Ar₁ | CH₂NH | H | CH/ CH | Et | 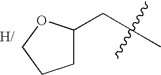 | 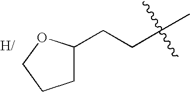 | | 625 | 6.52 (D) | NMR | |
| 60 | Ar₁ | CH₂NH | H | CH/ CH | Et | H/—CH₂CH₂N(i-Pr)₂ | H/—CH₂CH₂N(i-Pr)₂ | | 711 | 5.12 (D) | | |
| 61 | Ar₁ | CH₂NH | H | CH/ CH | Et | 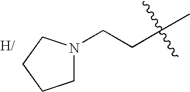 | 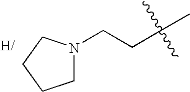 | | 651 | | 130- 135° C./ NMR | cf.ex.8 dihydro- chloride |
| 62 | Ar₁ | CH₂NH | H | N/ CH | Et | 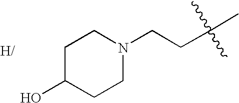 | 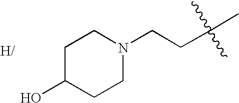 | | 712 | 4.43 (A) | | |
| 63 | Ar₁ | CH₂NH | H | N/ CH | Et | 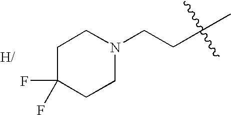 | 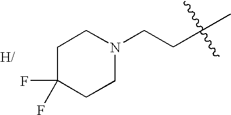 | | 752 | 5.36 (A) | | |
| 64 | Ar₁ | CH₂NH | H | N/ CH | Et | H/H | H/—C(CH₂OH)₃ | | 562 | 5.32 (A) | | |

abbreviations: n-Bu: n-butyl; t-Bu: tert-butyl; i-Pr: isopropyl
¹in L, NH, NMe or 0 is linked to the C═O group
²LC: liquid chromatography and tr: retention time
³Mp: melting point
⁴for all the compounds, x = 0 except for compound No. 4

The compounds of Table I have the following chemical nomenclature determined using the AUTONOM® software:
2-Amino-1-ethyl-4-oxo-7-[4-(3-pyridin-3-ylmethylureido)phenyl]-1,4-dihydro[1,8]naphthyridine-3-carboxylic acid methylamide (No. 1)
2-Amino-1-ethyl-4-oxo-7-[4-(3-pyridin-3-ylureido)phenyl]-1,4-dihydro[1,8]naphthyridine-3-carboxylic acid methylamide (No. 2)
2-Amino-1-ethyl-4-oxo-7-{4-[3-(2-pyridin-3-ylethyl)ureido]phenyl}-1,4-dihydro[1,8]naphthyridine-3-carboxylic acid methylamide (No. 3)
2-Amino-1-ethyl-7-[3-fluoro-4-(3-pyridin-3-ylmethylureido)phenyl]-4-oxo-1,4-dihydro[1,8]naphthyridine-3-carboxylic acid methylamide (No. 4)
2-Amino-7-[4-(3-(6-aminopyridin-3-ylmethylureido)phenyl]-1-ethyl-4-oxo-1,4-dihydro[1,8]naphthyridine-3-carboxylic acid methylamide (No. 5)
2-Amino-1-methyl-4-oxo-7-[4-(3-pyridin-3-ylmethyl ureido)phenyl]-1,4-dihydro[1,8]naphthyridine-3-carboxylic acid methylamide (No. 6)
2-Amino-1-ethyl-4-oxo-7-[4-((E)-3-pyridin-3-yl-acryloylamino)phenyl]-1,4-dihydro[1,8]naphthyridine-3-carboxylic acid methylamide (No. 7)
2-Amino-1-(2-dimethylamino-ethyl)-4-oxo-7-[4-(3-pyridin-3-ylmethylureido)phenyl]-1,4-dihydro[1,8]naphthyridine-3-carboxylic acid methylamide (No. 8)
2-Amino-1-ethyl-4-oxo-7-[4-(3-pyridin-3-ylmethylureido)phenyl]-1,4-dihydro[1,8]naphthyridine-3-carboxylic acid amide (No. 9)
2-Amino-1-ethyl-4-oxo-7-[4-(3-pyridin-3-ylmethylureido)phenyl]-1,4-dihydro[1,8]naphthyridine-3-carboxylic acid ethyl ester (No. 10)
2-Amino-7-{4-[3-(3-amino-benzyl)ureido]phenyl}-1-ethyl-4-oxo-1,4-dihydro[1,8]naphthyridine-3-carboxylic acid methylamide (No. 11)
2-Amino-1-ethyl-7-[4-(3-methyl-3-pyridin-3-ylmethylureido)phenyl]-4-oxo-1,4-dihydro[1,8]naphthyridine-3-carboxylic acid methylamide (No. 12)
2-Amino-1-ethyl-4-oxo-7-[4-(3-pyridin-3-ylmethylureido)phenyl]-1,4-dihydro[1,8]naphthyridine-3-carboxylic acid (No. 13)
2-Amino-1-ethyl-4-oxo-7-[4-(3-pyridin-3-ylmethylureido)phenyl]-1,4-dihydro[1,8]naphthyridine-3-carboxylic acid dimethylamide (No. 14)
2-Amino-1-ethyl-4-oxo-7-[4-(3-pyridin-3-ylmethylureido)phenyl]-1,4-dihydro[1,8]naphthyridine-3-carboxylic acid (2-dimethylaminoethyl)amide (No. 15)
{1-[4-(7-Amino-8-ethyl-6-methylcarbamoyl-5-oxo-5,8-dihydro[1,8]naphthyridin-2-yl)phenylcarbamoyl]-2-pyridin-3-ylethyl}carbamic acid tert-butyl ester (No. 16)
2-Amino-1-ethyl-4-oxo-7-[4-(3-thiazol-5-ylmethylureido)phenyl]-1,4-dihydro[1,8]naphthyridine-3-carboxylic acid methylamide (No. 17)
[4-(7-Amino-8-ethyl-6-methylcarbamoyl-5-oxo-5,8-dihydro[1,8]naphthyridin-2-yl)phenyl]carbamic acid pyridin-3-ylmethyl ester (No. 18)
2-Amino-1-ethyl-7-[4-(1-methyl-3-pyridin-3-ylmethylureido)phenyl]-4-oxo-1,4-dihydro[1,8]naphthyridine-3-carboxylic acid methylamide (No. 19)
[6-((2-amino-1-ethyl-4-oxo-7-[4-(3-pyridin-3-ylmethylureido)phenyl]-1,4-dihydro[1,8]naphthyridine-3-carbonyl)amino)hexyl]carbamic acid tert-butyl ester (No. 20)
2-Amino-7-{4-[3-(6-aminopyridin-3-ylmethyl)ureido]phenyl}-1-ethyl-4-oxo-1,4-dihydro[1,8]naphthyridine-3-carboxylic acid methylamide (No. 21)
2-(2-Dimethylaminoethylamino)-1-ethyl-4-oxo-7-[4-(3-pyridin-3-ylmethylureido)phenyl]-1,4-dihydro[1,8]naphthyridine-3-carboxylic acid (2-dimethylaminoethyl)amide (No. 22)
2-Amino-1-ethyl-7-[4-(3-methyl-3-pyridin-3-ylmethylureido)phenyl]-4-oxo-1,4-dihydro[1,8]naphthyridine-3-carboxylic acid amide (No. 23)
(6-{2-Amino-3-methylcarbamoyl-4-oxo-7-[4-(3-pyridin-3-ylmethylureido)phenyl]-4H-[1,8]naphthyridin-1-yl}-hexyl)carbamic acid tert-butyl ester (No. 24)
2-Amino-1-(6-aminohexyl)-4-oxo-7-[4-(3-pyridin-3-ylmethylureido)phenyl]-1,4-dihydro[1,8]naphthyridine-3-carboxylic acid methylamide (No. 25)
2-Amino-1-ethyl-4-oxo-7-[4-(3-pyridin-3-ylmethylureido)phenyl]-1,4-dihydro[1,8]naphthyridine-3-carboxylic acid (6-aminohexyl)amide (No. 26)
2-Amino-1-ethyl-4-oxo-7-[4-(3-pyridin-3-ylmethylureido)phenyl]-1,4-dihydroquinoline-3-carboxylic acid methylamide (No. 27)
1-Ethyl-2-methylamino-4-oxo-7-[4-(3-pyridin-3-ylmethylureido)phenyl]-1,4-dihydro[1,8]naphthyridine-3-carboxylic acid methylamide (No. 28)
1-{4-[7-Amino-8-ethyl-6-(4-methyl piperazine-1-carbonyl)-5-oxo-5,8-dihydro[1,8]naphthyridin-2-yl]phenyl}-3-pyridin-3-ylmethylurea (No. 29)
2-Amino-1-ethyl-4-oxo-7-[4-(3-pyridin-3-ylmethylureido)phenyl]-1,4-dihydro[1,8]naphthyridine-3-carboxylic acid (2-morpholin-4-ylethyl)amide (No. 30)
1-Ethyl-2-(2-morpholin-4-ylethylamino)-4-oxo-7-[4-(3-pyridin-3-ylmethylureido)phenyl]-1,4-dihydro[1,8]naphthyridine-3-carboxylic acid (2-morpholin-4-ylethyl)amide (No. 31)
2-Amino-1-ethyl-4-oxo-7-[4-(3-pyridin-3-ylmethylureido)phenyl]-1,4-dihydro[1,8]naphthyridine-3-carboxylic acid [2-(2-{2-[2-(2-{2-[2-(2-aminoethoxy)ethoxy]ethoxy}ethoxy)ethoxy]ethoxy}ethoxy)ethyl]amide (No. 32)
2-Amino-1-(2-morpholin-4-ylethyl)-4-oxo-7-[4-(3-pyridin-3-ylmethylureido)phenyl]-1,4-dihydro[1,8]naphthyridine-3-carboxylic acid methylamide (No. 33)
1-Ethyl-4-oxo-7-[4-(3-pyridin-3-ylmethylureido)phenyl]-2-(2-pyrrolidin-1-ylethylamino)-1,4-dihydro[1,8]naphthyridine-3-carboxylic acid (2-pyrrolidin-1-ylethyl)amide (No. 34)
2-Amino-1-(2-dimethylaminoethyl)-4-oxo-7-[4-(3-pyridin-3-ylmethylureido)phenyl]-1,4-dihydro[1,8]naphthyridine-3-carboxylic acid (2-dimethylaminoethyl)amide (No. 35)
2-Amino-1-ethyl-4-oxo-7-[4-(3-pyridin-3-ylmethylureido)phenyl]-1,4-dihydro[1,8]naphthyridine-3-carboxylic acid (2-pyrrolidin-1-ylethyl)amide (No. 36)
2-Amino-1-(2-morpholin-4-ylethyl)-4-oxo-7-[4-(3-pyridin-3-ylmethylureido)phenyl]-1,4-dihydro[1,8]naphthyridine-3-carboxylic acid amide (No. 37)
2-Amino-7-[4-(3-methyl-3-pyridin-3-ylmethylureido)phenyl]-1-(2-morpholin-4-ylethyl)-4-oxo-1,4-dihydro[1,8]naphthyridine-3-carboxylic acid methylamide (No. 38)
2-Amino-1-ethyl-7-[4-(3-methyl-3-pyridin-3-ylmethylureido)phenyl]-4-oxo-1,4-dihydro[1,8]naphthyridine-3-carboxylic acid (2-morpholin-4-ylethyl)amide (No. 39)
2-Amino-1-ethyl-4-oxo-7-[4-(3-pyridin-3-ylmethylureido)phenyl]-1,4-dihydro[1,8]naphthyridine-3-carboxylic acid {3-[3-(3-aminopropylamino)propylamino]propyl}amide (No. 40)
2-Amino-1-(2-morpholin-4-ylethyl)-4-oxo-7-[4-(3-pyridin-3-ylmethylureido)phenyl]-1,4-dihydro[1,8]naphthyridine-3-carboxylic acid (2-aminoethyl)amide (No. 41)
1-Ethyl-4-oxo-2-(2-piperazin-1-ylethylamino)-7-[4-(3-pyridin-3-ylmethylureido)phenyl]-1,4-dihydro[1,8]naphthyridine-3-carboxylic acid (2-piperazin-1-ylethyl)amide (No. 42)
2-Amino-1-ethyl-4-oxo-7-[4-(3-pyridin-3-ylmethylureido)phenyl]-1,4-dihydro[1,8]naphthyridine-3-carboxylic acid (2-piperazin-1-ylethyl)amide (No. 43)
1-Ethyl-4-oxo-2-[(piperidin-4-ylmethyl)-amino]-7-[4-(3-pyridin-3-ylmethylureido)phenyl]-1,4-dihydro[1,8]naphthyridine-3-carboxylic acid (piperidin-4-ylmethyl)amide (No. 44)
1-Ethyl-7-[4-(3-methyl-3-pyridin-3-ylmethylureido)phenyl]-2-(2-morpholin-4-ylethylamino)-4-oxo-1,4-dihydro[1,8]naphthyridine-3-carboxylic acid (2-morpholin-4-ylethyl)amide (No. 45)
1-Ethyl-2-[2-(4-methyl-piperazin-1-yl)-ethylamino]-4-oxo-7-[4-(3-pyridin-3-ylmethylureido)phenyl]-1,4-dihydro[1,8]naphthyridine-3-carboxylic acid [2-(4-methylpiperazin-1-yl)ethyl]amide (No. 46)
2-Amino-4-oxo-7-[4-(3-pyridin-3-ylmethyl ureido)phenyl]-1-(2-pyrrolidin-1-ylethyl)-1,4-dihydro[1,8]naphthyridine-3-carboxylic acid methylamide (No. 47)
2-Amino-1-ethyl-7-[4-(3-methyl-3-pyridin-3-ylmethyl ureido)phenyl]-4-oxo-1,4-dihydro[1,8]naphthyridine-3-carboxylic acid (2-piperazin-1-ylethyl)amide (No. 48)
2-Amino-1-ethyl-4-oxo-7-[4-(3-pyridin-3-ylmethylureido)phenyl]-1,4-dihydro[1,8]naphthyridine-3-carboxylic acid [2-(4-methyl-piperazin-1-yl)ethyl]amide (No. 49)
2-(2-Aminoethylamino)-1-ethyl-4-oxo-7-[4-(3-pyridin-3-ylmethylureido)phenyl]-1,4-dihydro[1,8]naphthyridine-3-carboxylic acid (2-aminoethyl)amide (No. 50)
2-Amino-1-ethyl-4-oxo-7-[4-(3-pyridin-3-ylmethylureido)phenyl]-1,4-dihydro[1,8]naphthyridine-3-carboxylic acid (piperidin-4-ylmethyl)amide (No. 51)
2-Amino-4-oxo-7-[4-(3-pyridin-3-ylmethyl ureido)phenyl]-1-(2-pyrrolidin-1-ylethyl)-1,4-dihydro[1,8]naphthyridine-3-carboxylic acid amide (No. 52)
7-Amino-8-ethyl-5-oxo-2-[4-(3-pyridin-3-ylmethylureido)phenyl]-5,8-dihydro-pyrido[2,3-d]pyrimidine-6-carboxylic acid methylamide (No. 53)
2-Amino-1-ethyl-4-oxo-7-[4-(3-pyridin-3-ylmethylureido)phenyl]-1,4-dihydro[1,8]naphthyridine-3-carboxylic acid (1-methyl-piperidin-4-ylmethyl)amide (No. 54)
2-(2-Diisopropylaminoethylamino)-1-ethyl-4-oxo-7-[4-(3-pyridin-3-ylmethylureido)phenyl]-1,4-dihydro[1,8]naphthyridine-3-carboxylic acid (2-diisopropylaminoethyl)amide (No. 55)
2-Amino-1-ethyl-4-oxo-7-[4-(3-pyridin-3-ylmethylureido)phenyl]-1,4-dihydroquinoline-3-carboxylic acid amide (No. 56)
1-Ethyl-2-[2-(2-methyl-pyrrolidin-1-yl)-ethylamino]-4-oxo-7-[4-(3-pyridin-3-ylmethylureido)phenyl]-1,4-dihydro[1,8]naphthyridine-3-carboxylic acid [2-(2-methylpyrrolidin-1-yl)ethyl]amide (No. 57)
1-Ethyl-2-[2-(2-methylpyrrolidin-1-yl)ethylamino]-4-oxo-7-[4-(3-pyridin-3-ylmethylureido)phenyl]-1,4-dihydro[1,8]naphthyridine-3-carboxylic acid [2-(2-methylpyrrolidin-1-yl)ethyl]amide (No. 58)
1-Ethyl-4-oxo-7-[4-(3-pyridin-3-ylmethylureido)phenyl]-2-[(tetrahydrofuran-2-ylmethyl)amino]-1,4-dihydroquinoline-3-carboxylic acid (tetrahydrofuran-2-ylmethyl)amide (No. 59)
2-(2-Diisopropylaminoethylamino)-1-ethyl-4-oxo-7-[4-(3-pyridin-3-ylmethylureido)phenyl]-1,4-dihydroquinoline-3-carboxylic acid (2-diisopropylaminoethyl)amide (No. 60)
1-Ethyl-4-oxo-7-[4-(3-pyridin-3-ylmethylureido)phenyl]-2-(2-pyrrolidin-1-ylethylamino)-1,4-dihydroquinoline-3-carboxylic acid (2-pyrrolidin-1-ylethyl)amide (No. 61)
1-Ethyl-2-[2-(4-hydroxypiperidin-1-yl)ethylamino]-4-oxo-7-[4-(3-pyridin-3-ylmethylureido)phenyl]-1,4-dihydro[1,8]naphthyridine-3-carboxylic acid [2-(4-hydroxypiperidin-1-yl)ethyl]amide (No. 62)
2-[2-(4,4-Difluoropiperidin-1-yl)ethylamino]-1-ethyl-4-oxo-7-[4-(3-pyridin-3-ylmethylureido)phenyl]-1,4-dihydro[1,8]naphthyridine-3-carboxylic acid [2-(4,4-difluoropiperidin-1-yl)ethyl]amide (No. 63)
2-Amino-1-ethyl-4-oxo-7-[4-(3-pyridin-3-ylmethylureido)phenyl]-1,4-dihydro[1,8]naphthyridine-3-carboxylic acid (2-hydroxy-1,1-bis-hydroxymethylethyl)amide (No. 64).

The proliferation and the cell viability were determined in a test using 3-(4,5-dimethylthiazol-2-yl)-5-(3-carboxymethoxyphenyl)-2-(4-sulfophenyl)-2H-tetrazolium (MTS) according to Fujishita T. et al. Oncology 2003, 64(4), 399-406 (see page 401). In this test, the mitochondrial capacity of the living cells for converting the MTS into a coloured compound, after incubation of the tested compound for 72 h, is measured. IC₅₀ (nM) denotes the concentration of compound which results in a 50% loss of proliferation and of cell viability.

The compounds of Table I were the subject of in vitro experimental tests on the HCT116 tumour line (ref. ATCC-CCL247). It was noted that the IC₅₀ values are between <0.1 and 1 μM for this line (see also the values given in the examples), indicating that said compounds have an anticancer activity.

We claim:

1. A compound of formula (I): 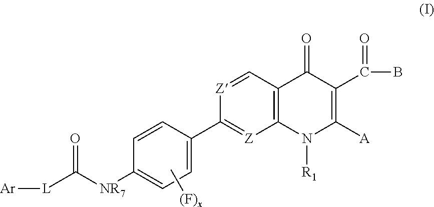
where:
R₁ represents:
a (C₃-C₇)cycloalkyl group or
a (C₁-C₆)alkyl group optionally substituted with an —NRₐR_{b}
group in which:
(i) Rₐ and R_{b} represent, independently of one another, a hydrogen atom or a (C₁-C₄)alkyl group, or
(ii) Rₐ and R_{b} form, together with the nitrogen atom to which they are attached, a heterocycloalkyl group which optionally comprises another heteroatom in the ring and which is optionally substituted;
or (iii) Rₐ represents a hydrogen atom and R_{b} represents a —C(═O)O(C₁-C₄)alkyl group;
A represents an —NR₂R₃ group;
B represents an —NR₄R₅ or —OR₆ group;
R₂ and R₃ are such that:
R₂ and R₃ represent, independently of one another, a hydrogen atom or a (C₁-C₆)alkyl group;
or R₂ represents a hydrogen atom and R₃ represents a (C₁-C₆)alkyl group substituted with:
an optionally substituted heterocycloalkyl group;
an —NR_{c}R_{d} group in which:
(i) R_{c} and R_{d} represent, independently of one another, a hydrogen atom or a (C₁-C₄)alkyl group;
or (ii) R_{c} and R_{d} form, together with the nitrogen atom to which they are attached, a heterocycloalkyl group which optionally comprises another heteroatom in the ring and which is optionally substituted;
R₄ and R₅ are such that:
R₄ and R₅ represent, independently of one another, a hydrogen atom or a (C₁-C₄)alkyl group, or form, together with the nitrogen atom to which they are attached, an optionally substituted heterocycloalkyl group;
or R₄ represents a hydrogen atom and R₅ represents a (C₁-C₆)alkyl group substituted with:
an optionally substituted heterocycloalkyl group;
an —NRₑR_{f} group in which:
(i) Rₑ and R_{f} represent a hydrogen atom or a (C₁-C₄)alkyl group;
or (ii) Rₑ and R_{f} form, together with the nitrogen atom to which they are attached, a heterocycloalkyl group which optionally comprises another heteroatom in the ring and which is optionally substituted;
or (iii) Rₑ represents a hydrogen atom and R_{f} represents a —C(═O)O(C₁-C₄)alkyl group;
or R₄ represents a hydrogen atom and R₅ represents one of the following groups: —C(CH₂OH)₃; —[(CH₂)₂O]ₘCH₂NH₂ or —[(CH₂)₃NH]ₘ—H in which m is an integer ranging from 3 to 10;
with the condition that, if R₂ represents a hydrogen atom and R₃ represents a (C₁-C₆)alkyl group substituted with a heterocycloalkyl group or an —NR_{c}R_{d} group, then R₄ and R₅ are respectively identical to R₂ and R₃;
R₆ represents a hydrogen atom or a (C₁-C₄)alkyl group;
Z represents N or CH;
Z′ represents N or CH if Z represents N, and CH if Z represents CH;
x is an integer ranging from 0 to 4;
R₇ represents a hydrogen atom or a (C₁-C₄)alkyl group;
L represents a —CH═CH—, —CH₂CH₂—, —CH₂CH[NHC(═O)O(C₁-C₄)alkyl)] or —(CH₂)ₙ—Y— group in which n is an integer ranging from 1 to 4 and the group Y (connected to C═O) represents an oxygen atom or an —NR₈— group in which R₈ represents a hydrogen atom or a (C₁-C₄)alkyl group; and
Ar represents a group chosen from: 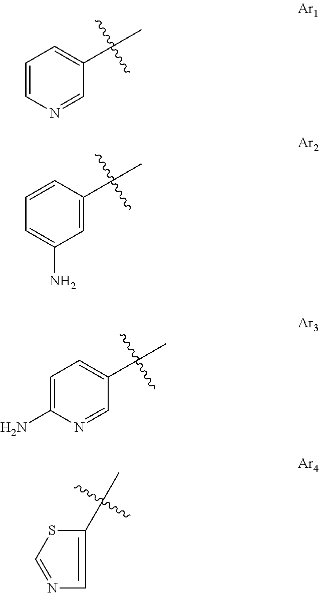
in the form of bases or of addition salts with an acid, and also in the form of hydrates or of solvates.

2. The compound according to claim 1, wherein the heterocycloalkyl group formed by Rₐ and R_{b} is the 4-morpholinyl 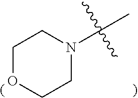
or pyrrolidinyl 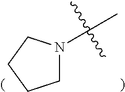
group.

3. The compound according to claim 1, wherein R₂ and R₃ both represent a hydrogen atom, or else R₂ represents a hydrogen atom and R₃:
a (C₁-C₆)alkyl group;
a (C₁-C₆)alkyl group substituted with an optionally substituted heterocycloalkyl group; or
a (C₁-C₆)alkyl group substituted with the —NR_{c}R_{d} group.

4. The compound according to claim 3, wherein R₃ represents a (C₁-C₆)alkyl group substituted with the 4-piperidinyl or 4-N-alkylpiperidinyl or 2-tetrahydrofuryl group.

5. The compound according to claim 3, wherein the —NR_{c}R_{d} group represents the 4-morpholinyl group, pyrrolidinyl group, piperazinyl group or N-alkylpiperazinyl group, piperidinyl group, 2-methylpyrrolidinyl group, 4-hydroxypiperidinyl group or 4,4′-difluoropiperidinyl group.

6. The compound according to claim 1, wherein R₄ and R₅ both represent a hydrogen atom or else a (C₁-C₆)alkyl group, or else R₄ represents a hydrogen atom and R₅:
a (C₁-C₆)alkyl group;
a (C₁-C₆)alkyl group substituted with an optionally substituted heterocycloalkyl group; or
a (C₁-C₆)alkyl group substituted with the —NRₑR_{f} group.

7. The compound according to claim 6, wherein R₅ represents a (C₁-C₆)alkyl group substituted with the 4-piperidinyl or 4-N-alkylpiperidinyl or 2-tetrahydrofuryl group.

8. The compound according to claim 6, wherein the —NRₑR_{f} group represents the 4-morpholinyl group, pyrrolidinyl group, piperazinyl group or N-alkylpiperazinyl group, piperidinyl group, 2-methylpyrrolidinyl group, 4-hydroxypiperidinyl group or 4,4′-difluoropiperidinyl group.

9. The compound according to claim 1, wherein L represents the —CH₂—NH—, —CH₂—O— or —CH═CH— group.

10. The compound according to claim 1, wherein the ring comprising Z and Z′ is one of the following:

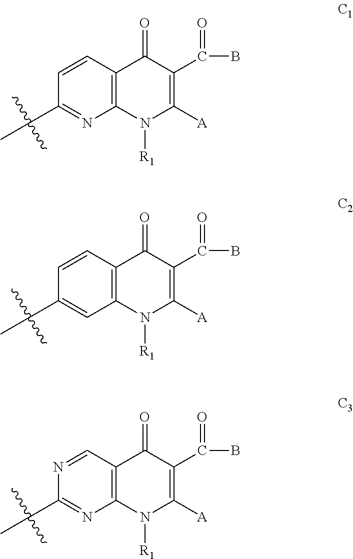

11. The compound according to claim 1, wherein:
R₁ represents a (C₁-C₆)alkyl group;
A represents an —NHR₃ group in which R₃ represents a hydrogen atom or a (C₁-C₄)alkyl group; and
B represents an —NR₄R₅ group in which R₄ represents a hydrogen atom or a (C₁-C₆)alkyl group and R₅ represents:
a hydrogen atom;
or a (C₁-C₆)alkyl group optionally substituted with:
an optionally substituted heterocycloalkyl group;
an —NRₑR_{f} group as defined in claim 1;
or one of the following groups: —C(CH₂OH)₃; —[(CH₂)₂O]ₘCH₂NH₂ or —[(CH₂)₃NH]ₘ—H in which m is an integer ranging from 3 to 10.

12. The compound according to claim 1, wherein:
R₁ represents a (C₁-C₆)alkyl group substituted with an —NRₐR_{b} group as defined in claim 1;
A represents an —NH₂ group; and
B represents an —NR₄R₅ group in which R₄ represents a hydrogen atom or a (C₁-C₆)alkyl group.

13. A compound of formula (II): 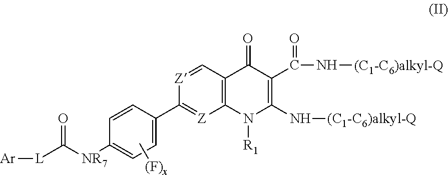
where:
Ar, L, R₇, Z, Z′ and x are as defined in claim 1;
R₁ represents a (C₁-C₆)alkyl group; and
Q denotes an optionally substituted heterocycloalkyl group or else the —NR_{c}R_{d} group as defined in claim 1.

14. The compound of claim 1 or 13, wherein:
Ar represents the Ar₁ group;
and/or R₇ represents a hydrogen atom;
and/or L represents CH₂NH;
and/or Z and Z′ represent respectively N and CH.

15. A compound which is:
2-Amino-1-ethyl-4-oxo-7-[4-(3-pyridin-3-ylmethylureido)phenyl]-1,4-dihydro[1,8]naphthyridine-3-carboxylic acid methylamide;
2-Amino-1-ethyl-4-oxo-7-[4-(3-pyridin-3-ylureido)phenyl]-1,4-dihydro[1,8]naphthyridine-3-carboxylic acid methylamide;
2-Amino-1-ethyl-4-oxo-7-{4-[3-(2-pyridin-3-ylethyl)ureido]phenyl}1,4-dihydro[1,8]naphthyridine-3-carboxylic acid methylamide;
2-Amino-1-ethyl-7-[3-fluoro-4-(3-pyridin-3-ylmethylureido)phenyl]-4-oxo-1,4-dihydro[1,8]naphthyridine-3-carboxylic acid methylamide;
2-Amino-7-[4-(3-(6-aminopyridin-3-ylmethylureido)phenyl]-1-ethyl-4-oxo-1,4-dihydro[1,8]naphthyridine-3-carboxylic acid methylamide;
2-Amino-1-methyl-4-oxo-7-[4-(3-pyridin-3-ylmethyl ureido)phenyl]-1,4-dihydro[1,8]naphthyridine-3-carboxylic acid methylamide;
2-Amino-1-ethyl-4-oxo-7-[4-((E)-3-pyridin-3-yl-acryloylamino)phenyl]-1,4-dihydro[1,8]naphthyridine-3-carboxylic acid methylamide;
2-Amino-1-(2-dimethylaminoethyl)-4-oxo-7-[4-(3-pyridin-3-ylmethylureido)phenyl]-1,4-dihydro[1,8]naphthyridine-3-carboxylic acid methylamide;
2-Amino-1-ethyl-4-oxo-7-[4-(3-pyridin-3-ylmethylureido)phenyl]-1,4-dihydro[1,8]naphthyridine-3-carboxylic acid amide;
2-Amino-1-ethyl-4-oxo-7-[4-(3-pyridin-3-ylmethylureido)phenyl]-1,4-dihydro[1,8]naphthyridine-3-carboxylic acid ethyl ester;
2-Amino-7-{4-[3-(3-aminobenzyl)ureido]phenyl}1-ethyl-4-oxo-1,4-dihydro[1,8]naphthyridine-3-carboxylic acid methylamide;
2-Amino-1-ethyl-7-[4-(3-methyl-3-pyridin-3-ylmethylureido)phenyl]-4-oxo-1,4-dihydro[1,8]naphthyridine-3-carboxylic acid methylamide;
2-Amino-1-ethyl-4-oxo-7-[4-(3-pyridin-3-ylmethylureido)phenyl]-1,4-dihydro[1,8]naphthyridine-3-carboxylic acid;
2-Amino-1-ethyl-4-oxo-7-[4-(3-pyridin-3-ylmethylureido)phenyl]-1,4-dihydro[1,8]naphthyridine-3-carboxylic acid dimethylamide;
2-Amino-1-ethyl-4-oxo-7-[4-(3-pyridin-3-ylmethylureido)phenyl]-1,4-dihydro[1,8]naphthyridine-3-carboxylic acid (2-dimethylaminoethyl)amide;
{1-[4-(7-Amino-8-ethyl-6-methylcarbamoyl-5-oxo-5,8-dihydro[1,8]naphthyridin-2-yl)phenylcarbamoyl]-2-pyridin-3-ylethyl}carbamic acid tert-butyl ester;
2-Amino-1-ethyl-4-oxo-7-[4-(3-thiazol-5-ylmethylureido)phenyl]-1,4-dihydro[1,8]naphthyridine-3-carboxylic acid methylamide;
[4-(7-Amino-8-ethyl-6-methylcarbamoyl-5-oxo-5,8-dihydro[1,8]naphthyridin-2-yl)phenyl]carbamic acid pyridin-3-ylmethyl ester;
2-Amino-1-ethyl-7-[4-(1-methyl-3-pyridin-3-ylmethylureido)phenyl]-4-oxo-1,4-dihydro[1,8]naphthyridine-3-carboxylic acid methylamide;
[6-((2-amino-1-ethyl-4-oxo-7-[4-(3-pyridin-3-ylmethylureido)phenyl]-1,4-dihydro[1,8]naphthyridine-3-carbonyl)amino)-hexyl]carbamic acid tert-butyl ester;
2-Amino-7-{4-[3-(6-aminopyridin-3-ylmethyl)ureido]phenyl}-1-ethyl-4-oxo-1,4-dihydro[1,8]naphthyridine-3-carboxylic acid methylamide;
2-(2-Dimethylaminoethylamino)-1-ethyl-4-oxo-7-[4-(3-pyridin-3-ylmethylureido)phenyl]-1,4-dihydro[1,8]naphthyridine-3-carboxylic acid (2-dimethylaminoethyl)amide;
2-Amino-1-ethyl-7-[4-(3-methyl-3-pyridin-3-ylmethylureido)phenyl]-4-oxo-1,4-dihydro[1,8]naphthyridine-3-carboxylic acid amide;
(6-{2-Amino-3-methylcarbamoyl-4-oxo-7-[4-(3-pyridin-3-ylmethylureido)phenyl]-4H-[1,8]naphthyridin-1-yl}hexyl)carbamic acid tert-butyl ester;
2-Amino-1-(6-aminohexyl)-4-oxo-7-[4-(3-pyridin-3-ylmethylureido)phenyl]-1,4-dihydro[1,8]naphthyridine-3-carboxylic acid methylamide;
2-Amino-1-ethyl-4-oxo-7-[4-(3-pyridin-3-ylmethylureido)phenyl]-1,4-dihydro[1,8]naphthyridine-3-carboxylic acid (6-aminohexyl)amide;
2-Amino-1-ethyl-4-oxo-7-[4-(3-pyridin-3-ylmethylureido)phenyl]-1,4-dihydroquinoline-3-carboxylic acid methylamide;
1-Ethyl-2-methylamino-4-oxo-7-[4-(3-pyridin-3-ylmethylureido)phenyl]-1,4-dihydro[1,8]naphthyridine-3-carboxylic acid methylamide;
1-{4-[7-Amino-8-ethyl-6-(4-methylpiperazine-1-carbonyl)-5-oxo-5,8-dihydro[1,8]naphthyridin-2-yl]phenyl}-3-pyridin-3-ylmethylurea;
2-Amino-1-ethyl-4-oxo-7-[4-(3-pyridin-3-ylmethylureido)phenyl]-1,4-dihydro[1,8]naphthyridine-3-carboxylic acid (2-morpholin-4-ylethyl)amide;
1-Ethyl-2-(2-morpholin-4-ylethylamino)-4-oxo-7-[4-(3-pyridin-3-ylmethylureido)phenyl]-1,4-dihydro[1,8]naphthyridine-3-carboxylic acid (2-morpholin-4-ylethyl)amide;
2-Amino-1-ethyl-4-oxo-7-[4-(3-pyridin-3-ylmethylureido)phenyl]-1,4-dihydro[1,8]naphthyridine-3-carboxylic acid [2-(2-{2-[2-(2-{2-[2-(2-aminoethoxy)ethoxy]ethoxy}ethoxy)ethoxy]ethoxy}ethoxy)ethyl]amide;
2-Amino-1-(2-morpholin-4-ylethyl)-4-oxo-7-[4-(3-pyridin-3-ylmethylureido)phenyl]-1,4-dihydro[1,8]naphthyridine-3-carboxylic acid methylamide;
1-Ethyl-4-oxo-7-[4-(3-pyridin-3-ylmethylureido)phenyl]-2-(2-pyrrolidin-1-ylethylamino)-1,4-dihydro[1,8]naphthyridine-3-carboxylic acid (2-pyrrolidin-1-ylethyl)amide;
2-Amino-1-(2-dimethylaminoethyl)-4-oxo-7-[4-(3-pyridin-3-ylmethylureido)phenyl]-1,4-dihydro[1,8]naphthyridine-3-carboxylic acid (2-dimethylaminoethyl)amide;
2-Amino-1-ethyl-4-oxo-7-[4-(3-pyridin-3-ylmethylureido)phenyl]-1,4-dihydro[1,8]naphthyridine-3-carboxylic acid (2-pyrrolidin-1-ylethyl)amide;
2-Amino-1-(2-morpholin-4-ylethyl)-4-oxo-7-[4-(3-pyridin-3-ylmethylureido)phenyl]-1,4-dihydro[1,8]naphthyridine-3-carboxylic acid amide;
2-Amino-7-[4-(3-methyl-3-pyridin-3-ylmethylureido)phenyl]-1-(2-morpholin-4-ylethyl)-4-oxo-1,4-dihydro[1,8]naphthyridine-3-carboxylic acid methylamide;
2-Amino-1-ethyl-7-[4-(3-methyl-3-pyridin-3-ylmethylureido)phenyl]-4-oxo-1,4-dihydro[1,8]naphthyridine-3-carboxylic acid (2-morpholin-4-ylethyl)amide;
2-Amino-1-ethyl-4-oxo-7-[4-(3-pyridin-3-ylmethylureido)phenyl]-1,4-dihydro[1,8]naphthyridine-3-carboxylic acid {3-[3-(3-amino-propylamino)propylamino]propyl}amide;
2-Amino-1-(2-morpholin-4-ylethyl)-4-oxo-7-[4-(3-pyridin-3-ylmethylureido)phenyl]-1,4-dihydro[1,8]naphthyridine-3-carboxylic acid (2-aminoethyl)amide;
1-Ethyl-4-oxo-2-(2-piperazin-1-ylethylamino)-7-[4-(3-pyridin-3-ylmethylureido)phenyl]-1,4-dihydro[1,8]naphthyridine-3-carboxylic acid (2-piperazin-1-ylethyl)amide;
2-Amino-1-ethyl-4-oxo-7-[4-(3-pyridin-3-ylmethylureido)phenyl]-1,4-dihydro[1,8]naphthyridine-3-carboxylic acid (2-piperazin-1-ylethyl)amide;
1-Ethyl-4-oxo-2-[(piperidin-4-ylmethyl)amino]-7-[4-(3-pyridin-3-ylmethylureido)phenyl]-1,4-dihydro[1,8]naphthyridine-3-carboxylic acid (piperidin-4-ylmethyl)amide;
1-Ethyl-7-[4-(3-methyl-3-pyridin-3-ylmethylureido)phenyl]-2-(2-morpholin-4-ylethylamino)-4-oxo-1,4-dihydro[1,8]naphthyridine-3-carboxylic acid (2-morpholin-4-ylethyl)amide;
1-Ethyl-2-[2-(4-methylpiperazin-1-yl)ethylamino]-4-oxo-7-[4-(3-pyridin-3-ylmethylureido)phenyl]-1,4-dihydro[1,8]naphthyridine-3-carboxylic acid [2-(4-methylpiperazin-1-yl)ethyl]amide;
2-Amino-4-oxo-7-[4-(3-pyridin-3-ylmethylureido)phenyl]-1-(2-pyrrolidin-1-ylethyl)-1,4-dihydro[1,8]naphthyridine-3-carboxylic acid methylamide;
2-Amino-1-ethyl-7-[4-(3-methyl-3-pyridin-3-ylmethylureido)phenyl]-4-oxo-1,4-dihydro[1,8]naphthyridine-3-carboxylic acid (2-piperazin-1-ylethyl)amide;
2-Amino-1-ethyl-4-oxo-7-[4-(3-pyridin-3-ylmethylureido)phenyl]-1,4-dihydro[1,8]naphthyridine-3-carboxylic acid [2-(4-methylpiperazin-1-yl)ethyl]amide;
2-(2-Aminoethylamino)-1-ethyl-4-oxo-7-[4-(3-pyridin-3-ylmethylureido)phenyl]-1,4-dihydro[1,8]naphthyridine-3-carboxylic acid (2-aminoethyl)amide;
2-Amino-1-ethyl-4-oxo-7-[4-(3-pyridin-3-ylmethylureido)phenyl]-1,4-dihydro[1,8]naphthyridine-3-carboxylic acid (piperidin-4-ylmethyl)amide;
2-Amino-4-oxo-7-[4-(3-pyridin-3-ylmethylureido)phenyl]-1-(2-pyrrolidin-1-ylethyl)-1,4-dihydro[1,8]naphthyridine-3-carboxylic acid amide;
7-Amino-8-ethyl-5-oxo-2-[4-(3-pyridin-3-ylmethylureido)phenyl]-5,8-dihydropyrido[2,3-d]pyrimidine-6-carboxylic acid methylamide;
2-Amino-1-ethyl-4-oxo-7-[4-(3-pyridin-3-ylmethylureido)phenyl]-1,4-dihydro[1,8]naphthyridine-3-carboxylic acid (1-methylpiperidin-4-ylmethyl)amide;
2-(2-Diisopropylaminoethylamino)-1-ethyl-4-oxo-7-[4-(3-pyridin-3-ylmethylureido)phenyl]-1,4-dihydro[1,8]naphthyridine-3-carboxylic acid (2-diisopropylaminoethyl)amide;
2-Amino-1-ethyl-4-oxo-7-[4-(3-pyridin-3-ylmethylureido)phenyl]-1,4-dihydroquinoline-3-carboxylic acid amide;
1-Ethyl-2-[2-(2-methylpyrrolidin-1-yl)ethylamino]-4-oxo-7-[4-(3-pyridin-3-ylmethylureido)phenyl]-1,4-dihydro[1,8]naphthyridine-3-carboxylic acid [2-(2-methylpyrrolidin-1-yl)ethyl]amide;
1-Ethyl-2-[2-(2-methylpyrrolidin-1-yl)ethylamino]-4-oxo-7-[4-(3-pyridin-3-ylmethylureido)phenyl]-1,4-dihydro[1,8]naphthyridine-3-carboxylic acid [2-(2-methylpyrrolidin-1-yl)ethyl]amide;
1-Ethyl-4-oxo-7-[4-(3-pyridin-3-ylmethylureido)phenyl]-2-[(tetrahydrofuran-2-ylmethyl)amino]-1,4-dihydroquinoline-3-carboxylic acid (tetrahydrofuran-2-ylmethyl)amide;
2-(2-Diisopropylaminoethylamino)-1-ethyl-4-oxo-7-[4-(3-pyridin-3-ylmethylureido)phenyl]-1,4-dihydroquinoline-3-carboxylic acid (2-diisopropylaminoethyl)amide;
1-Ethyl-4-oxo-7-[4-(3-pyridin-3-ylmethylureido)phenyl]-2-(2-pyrrolidin-1-ylethylamino)-1,4-dihydroquinoline-3-carboxylic acid (2-pyrrolidin-1-ylethyl)amide;
1-Ethyl-2-[2-(4-hydroxypiperidin-1-yl)ethylamino]-4-oxo-7-[4-(3-pyridin-3-ylmethylureido)phenyl]-1,4-dihydro[1,8]naphthyridine-3-carboxylic acid [2-(4-hydroxypiperidin-1-yl)ethyl]amide;
2-[2-(4,4-Difluoropiperidin-1-yl)ethylamino]-1-ethyl-4-oxo-7-[4-(3-pyridin-3-ylmethylureido)phenyl]-1,4-dihydro[1,8]naphthyridine-3-carboxylic acid [2-(4,4-difluoropiperidin-1-yl)ethyl]amide;
2-Amino-1-ethyl-4-oxo-7-[4-(3-pyridin-3-ylmethylureido)phenyl]-1,4-dihydro[1,8]naphthyridine-3-carboxylic acid (2-hydroxy-1,1-bishydroxymethylethyl)amide; or
a base, addition salt with an acid, hydrate or solvate thereof.

16. A process for preparing a compound of formula (I): 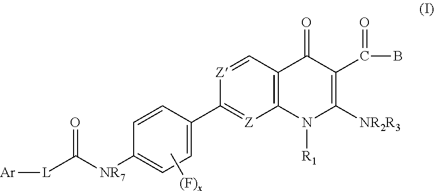
comprising coupling, in the presence of a palladium complex and optionally of a base, the following compounds P₁ and P₂: 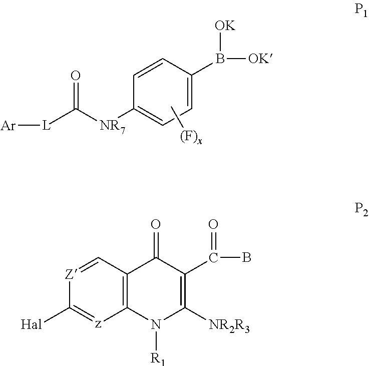
in which formulae:
Ar, L, R₇, Z, Z′, R₁, B and x are as defined in claim 1;
R₂ and R₃ represent, independently of one another, a hydrogen atom or a (C₁-C₆)alkyl group;
Hal represents a chlorine, bromine or iodine atom;
K and K′ represent a hydrogen atom, or an alkyl or aryl group, optionally linked to one another so as to form, together with the boron atom and the two oxygen atoms, a 5- to 7-membered ring optionally substituted with one or more alkyl group(s) or optionally joined to a phenyl group.

17. The process according to claim 16, wherein the —B(OK)(OK′) group is selected from the group consisting of

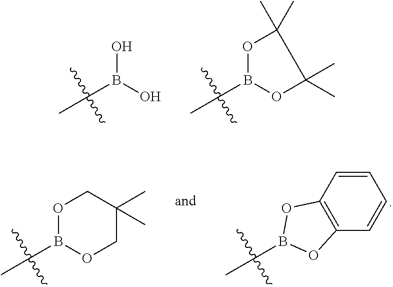

18. The process according to claim 16, wherein the palladium complex is in the oxidation state (0) or (II).

19. A process for preparing a compound of formula (I) 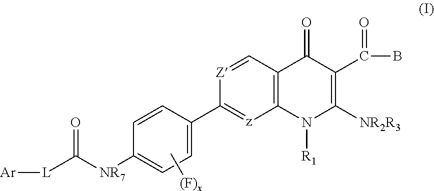
comprising coupling the compound P₃: 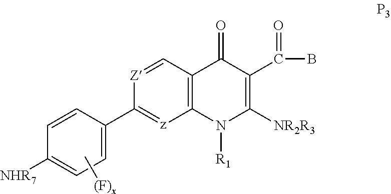
with the compound:
P₄ of formula Ar—(CH₂)ₙ—YH in the presence of an agent for introducing the “C═O” unit;
P₅ of formula Ar—CH═CH—COOH; and
P₆ of formula Ar—CH₂CH₂—COOH;
in which formulae Ar, L, R₇, Z, Z′, R₁, R₂, R₃, B, x, Y and n are as defined in claim 1.

20. The process according to claim 19, wherein the agent for introducing the “C═O” unit is phosgene, triphosgene or N,N′-disuccinimidyl carbonate.

21. The process according to claim 19, wherein the coupling between P₃ and P₅ or P₆ is carried out in the presence of an acid activator.

22. A process for preparing a compound of formula: 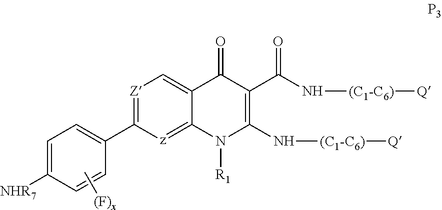
comprising reacting P₂₁ of formula: 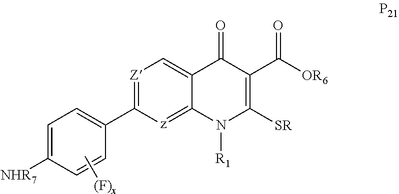
with H₂N—(C₁-C₆)alkyl-Q′, in which formulae:
R₁ represents a (C₁-C₆)alkyl group;
R₆ represents a hydrogen atom or a (C₁-C₄)alkyl group;
Z represents N or CH;
Z′ represents N or CH if Z represents N, and CH if Z represents CH;
x is an integer ranging from 0 to 4;
R₇ represents a hydrogen atom or a (C₁-C₄)alkyl group;
R represents a (C₁-C₄)alkyl group; and
Q′ represents an optionally substituted heterocycloalkyl group or else the —NR_{c}R_{d} group as defined in claim 1, except when Q represents an —NH₂ group, in which case Q′ represents —NH—PG in which PG represents a protective group for the amine function.

23. A pharmaceutical composition comprising a compound of claim 1 and at least one pharmaceutically acceptable excipient.

24. A method of treating a patient with cancer comprising administering to said patient a compound of claim 1.

25. A compound of formula P₂₁: 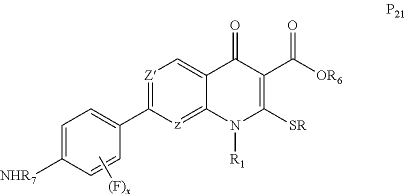
where:
R₁ represents a (C₁-C₄)alkyl group;
R₆ represents a hydrogen atom or a (C₁-C₄)alkyl group;
Z represents N or CH;
Z′ represents N or CH if Z represents N, and CH if Z represents CH;
x is an integer ranging from 0 to 4;
R₇ represents a hydrogen atom or a (C₁-C₄)alkyl group; and
R represents a (C₁-C₄)alkyl group.

26. A compound of formula: 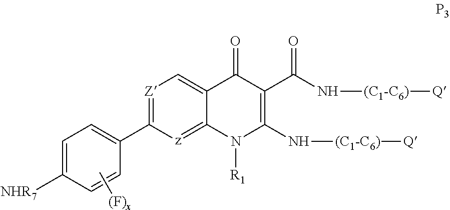
where:
R₁ represents a (C₁-C₆)alkyl;
Z represents N or CH;
Z′ represents N or CH if Z represents N, and CH if Z represents CH;
x is an integer ranging from 0 to 4;
R₇ represents a hydrogen atom or a (C₁-C₄)alkyl group; and
Q′ represents an optionally substituted heterocycloalkyl group or else the —NR_{c}R_{d} group as defined in claim 1, except when Q represents an —NH₂ group, in which case Q′ represents —NH—PG in which PG represents a protective group for the amine function.

27. A process for preparing a compound according to claim 1, said process comprising using as an intermediate a compound of formula P₂₁: 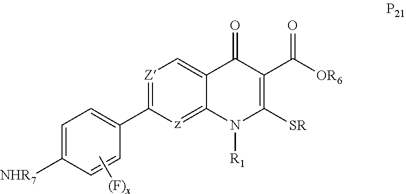
where:
R₁ represents a (C₁-C₄)alkyl group;
R₆ represents a hydrogen atom or a (C₁-C₄)alkyl group;
Z represents N or CH;
Z′ represents N or CH if Z represents N, and CH if Z represents CH;
x is an integer ranging from 0 to 4;
R₇ represents a hydrogen atom or a (C₁-C₄)alkyl group; and
R represents a (C₁-C₄)alkyl group.

28. A process for preparing a compound according to claim 1, said process comprising using as an intermediate a compound of compound of formula 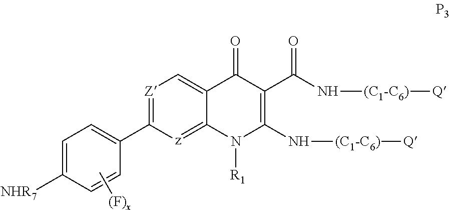
where:
R₁ represents a (C₁-C₆)alkyl;
Z represents N or CH;
Z′ represents N or CH if Z represents N, and CH if Z represents CH;
x is an integer ranging from 0 to 4;
R₇ represents a hydrogen atom or a (C₁-C₄)alkyl group; and
Q′ represents an optionally substituted heterocycloalkyl group or else an —NR_{c}R_{d} group in which:
(i) R_{c} and R_{d} represent, independently of one another, a hydrogen atom or a (C₁-C₄)alkyl group;
or (ii) R_{c} and R_{d} form, together with the nitrogen atom to which they are attached, a heterocycloalkyl group which optionally comprises another heteroatom in the ring and which is optionally substituted;
except when Q represents an —NH₂ group, in which case Q′ represents —NH—PG in which PG represents a protective group for the amine function.
